SEQUENCE LISTING (71 sequences)

SEQ ID NO: 1 - DNA, 32 bp, Artificial Sequence
  Synthetic polynucleotide
cttgtatatagacggtaaaataaacaccaaga

SEQ ID NO: 2 - DNA, 23 bp, Artificial Sequence
  Synthetic polynucleotide
aggcaccagaagtcatcagaatg

SEQ ID NO: 3 - DNA, 33 bp, Artificial Sequence
  Synthetic polynucleotide
taaacaccaagacgtggtaaatatttacctggt

SEQ ID NO: 4 - DNA, 23 bp, Artificial Sequence
  Synthetic polynucleotide
ccctgacaacatagttggaatca

SEQ ID NO: 5 - DNA, 25 bp, Artificial Sequence
  Synthetic polynucleotide
actccaggataatacacatcacagt

SEQ ID NO: 6 - DNA, 27 bp, Artificial Sequence
  Synthetic polynucleotide
tggaatcactcatgatatctcgagcca

SEQ ID NO: 7 - DNA, 17 bp, Artificial Sequence
  Synthetic polynucleotide
agcagtctctgcctcgc

SEQ ID NO: 8 - DNA, 23 bp, Artificial Sequence
  Synthetic polynucleotide
agaagattcggcagaactaagca

SEQ ID NO: 9 - DNA, 21 bp, Artificial Sequence
  Synthetic polynucleotide
cctcgccaagcggctcatgtt

SEQ ID NO: 10 - DNA, 23 bp, Artificial Sequence
  Synthetic polynucleotide
gcttgtgagtggatgggtaaaac

SEQ ID NO: 11 - DNA, 22 bp, Artificial Sequence
  Synthetic polynucleotide
tgtgttgagatggacgcctatt

SEQ ID NO: 12 - DNA, 31 bp, Artificial Sequence
  Synthetic polynucleotide
tgggtaaaacctatcatcataggtcgtcatg

SEQ ID NO: 13 - DNA, 24 bp, Artificial Sequence
  Synthetic polynucleotide
agaagatgtggaaaagtcccaatg

SEQ ID NO: 14 - DNA, 18 bp, Artificial Sequence
  Synthetic polynucleotide
gtgcccaggtcagtggat

SEQ ID NO: 15 - DNA, 26 bp, Artificial Sequence
  Synthetic polynucleotide
tcccaatggaactatccggaacatcc

SEQ ID NO: 16 - DNA, 17 bp, Artificial Sequence
  Synthetic polynucleotide
ctggtcgccatgggcgt

SEQ ID NO: 17 - DNA, 24 bp, Artificial Sequence
  Synthetic polynucleotide
tgaagaagatgtggaaaagtccca

SEQ ID NO: 18 - DNA, 20 bp, Artificial Sequence
  Synthetic polynucleotide
ggcgtgcctgccaatggtga

SEQ ID NO: 19 - DNA, 32 bp, Artificial Sequence
  Synthetic polynucleotide
tcctttaaccacataattagaatcattcttga

SEQ ID NO: 20 - DNA, 32 bp, Artificial Sequence
  Synthetic polynucleotide
agttagttttcactctttacaagttaaaatga

SEQ ID NO: 21 - DNA, 30 bp, Artificial Sequence
  Synthetic polynucleotide
atcattcttgatgtctctggctagaccaaa

SEQ ID NO: 22 - DNA, 32 bp, Artificial Sequence
  Synthetic polynucleotide
gtttaaactattttcttaaagagacttcctcc

SEQ ID NO: 23 - DNA, 32 bp, Artificial Sequence
  Synthetic polynucleotide
ttaaagtgtttggaattaaattacatctgagt

SEQ ID NO: 24 - DNA, 27 bp, Artificial Sequence
  Synthetic polynucleotide
acttcctccactgccagagatcttgaa

SEQ ID NO: 25 - DNA, 19 bp, Artificial Sequence
  Synthetic polynucleotide
ttgaggcctttcttaccca

SEQ ID NO: 26 - DNA, 19 bp, Artificial Sequence
  Synthetic polynucleotide
ggcttgtgggagaccttga

SEQ ID NO: 27 - DNA, 25 bp, Artificial Sequence
  Synthetic polynucleotide
tcttacccagaagcagaaggtggga

SEQ ID NO: 28 - DNA, 17 bp, Artificial Sequence
  Synthetic polynucleotide
agctgcaggttctgcat

SEQ ID NO: 29 - DNA, 20 bp, Artificial Sequence
  Synthetic polynucleotide
agccacccaactcttaaggc

SEQ ID NO: 30 - DNA, 25 bp, Artificial Sequence
  Synthetic polynucleotide
ctgcatgagtgcaactctccgtaca

SEQ ID NO: 31 - DNA, 19 bp, Artificial Sequence
  Synthetic polynucleotide
cgctgaccccaaagtcaca

SEQ ID NO: 32 - DNA, 27 bp, Artificial Sequence
  Synthetic polynucleotide
agttccctccttttctattttctcttc

SEQ ID NO: 33 - DNA, 26 bp, Artificial Sequence
  Synthetic polynucleotide
aaagtcacagagcttgatctccccac

SEQ ID NO: 34 - DNA, 16 bp, Artificial Sequence
  Synthetic polynucleotide
ttcgccgaccttggct

SEQ ID NO: 35 - DNA, 22 bp, Artificial Sequence
  Synthetic polynucleotide
agtctccctaggtagctaaccc

SEQ ID NO: 36 - DNA, 25 bp, Artificial Sequence
  Synthetic polynucleotide
ttggctttctgggtgagaaaggctt

SEQ ID NO: 37 - DNA, 17 bp, Artificial Sequence
  Synthetic polynucleotide
ctgcaggtcctgcacga

SEQ ID NO: 38 - DNA, 19 bp, Artificial Sequence
  Synthetic polynucleotide
ggacgcactcaccatgtgt

SEQ ID NO: 39 - DNA, 23 bp, Artificial Sequence
  Synthetic polynucleotide
ctgcacgaatgcaactcgccgta

SEQ ID NO: 40 - DNA, 21 bp, Artificial Sequence
  Synthetic polynucleotide
catcctcgtgaactctagagg

SEQ ID NO: 41 - DNA, 20 bp, Artificial Sequence
  Synthetic polynucleotide
gggactcacagccatgtagg

SEQ ID NO: 42 - DNA, 26 bp, Artificial Sequence
  Synthetic polynucleotide
tctagaggggagatcaagctgtgtga

SEQ ID NO: 43 - DNA, 19 bp, Artificial Sequence
  Synthetic polynucleotide
aacaccttcatcatccgct

SEQ ID NO: 44 - DNA, 17 bp, Artificial Sequence
  Synthetic polynucleotide
ccatccccgtgtccctc

SEQ ID NO: 45 - DNA, 21 bp, Artificial Sequence
  Synthetic polynucleotide
cgctgcctgcagtggaccact

SEQ ID NO: 46 - DNA, 24 bp, Artificial Sequence
  Synthetic polynucleotide
tcaaaaggaagtatcttggcctcc

SEQ ID NO: 47 - DNA, 27 bp, Artificial Sequence
  Synthetic polynucleotide
ccagtgttgtaggacatatattgtacc

SEQ ID NO: 48 - DNA, 34 bp, Artificial Sequence
  Synthetic polynucleotide
gcctccagttttttatatattctcctacatgagg

SEQ ID NO: 49 - DNA, 19 bp, Artificial Sequence
  Synthetic polynucleotide
cagtgcgcttttcccaaca

SEQ ID NO: 50 - DNA, 26 bp, Artificial Sequence
  Synthetic polynucleotide
gctttaaagtactgtagatgtggctc

SEQ ID NO: 51 - DNA, 22 bp, Artificial Sequence
  Synthetic polynucleotide
cccaacaccacctgctccaacc

SEQ ID NO: 52 - DNA, 23 bp, Artificial Sequence
  Synthetic polynucleotide
ttgttggacatactggatacagc

SEQ ID NO: 53 - DNA, 24 bp, Artificial Sequence
  Synthetic polynucleotide
ggttaatatccgcaaatgacttgc

SEQ ID NO: 54 - DNA, 28 bp, Artificial Sequence
  Synthetic polynucleotide
gatacagctggacaagaagagtacagtg

SEQ ID NO: 55 - DNA, 20 bp, Artificial Sequence
  Synthetic polynucleotide
gtcaaggcactcttgcctac

SEQ ID NO: 56 - DNA, 30 bp, Artificial Sequence
  Synthetic polynucleotide
tgtattaaccttatgtgtgacatgttctaa

SEQ ID NO: 57 - DNA, 20 bp, Artificial Sequence
  Synthetic polynucleotide
tgcctacgccaccagctcca

SEQ ID NO: 58 - DNA, 23 bp, Artificial Sequence
  Synthetic polynucleotide
tcttggatattctcgacacagca

SEQ ID NO: 59 - DNA, 32 bp, Artificial Sequence
  Synthetic polynucleotide
ttatattcaatttaaacccacctataatggtg

SEQ ID NO: 60 - DNA, 26 bp, Artificial Sequence
  Synthetic polynucleotide
cacagcaggtcaagaggagtacagtg

SEQ ID NO: 61 - DNA, 24 bp, Artificial Sequence
  Synthetic polynucleotide
caatttctacacgagatcctctct

SEQ ID NO: 62 - DNA, 26 bp, Artificial Sequence
  Synthetic polynucleotide
ggtatggtaaaaacatgctgagatca

SEQ ID NO: 63 - DNA, 27 bp, Artificial Sequence
  Synthetic polynucleotide
atcctctctctgaaatcactgagcagg

SEQ ID NO: 64 - DNA, 30 bp, Artificial Sequence
  Synthetic polynucleotide
ttggagtatttcatgaaacaaatgaatgat

SEQ ID NO: 65 - DNA, 20 bp, Artificial Sequence
  Synthetic polynucleotide
cagtgcagtgtggaatccag

SEQ ID NO: 66 - DNA, 27 bp, Artificial Sequence
  Synthetic polynucleotide
caaatgaatgatgcacatcatggtggc

SEQ ID NO: 67 - DNA, 19 bp, Artificial Sequence
  Synthetic polynucleotide
ggacccactccatcgagat

SEQ ID NO: 68 - DNA, 33 bp, Artificial Sequence
  Synthetic polynucleotide
ttacttactacacctcagatatatttcttcatg

SEQ ID NO: 69 - DNA, 31 bp, Artificial Sequence
  Synthetic polynucleotide
ccatcgagatttcactgtagctagaccaaaa

SEQ ID NO: 70 - DNA, 17 bp, Artificial Sequence
  Synthetic polynucleotide
caattcggtctccagtg

SEQ ID NO: 71 - DNA, 17 bp, Artificial Sequence
  Synthetic polynucleotide
caattcggtctcccact

(19) United States
(12) Patent Application Publication
(10) Pub. No.: US 2022/0411863 A1
(43) Pub. Date: Dec. 29, 2022

(54) LINEAR DNA ASSEMBLY FOR NANOPORE SEQUENCING

(71) Applicant: William Marsh Rice University, Houston, TX (US)

(72) Inventors: David Yu ZHANG, Houston, TX (US); Deepak THIRUNAVUKARASU, Houston, TX (US); Yuxuan CHENG, Houston, TX (US); Ping SONG, Houston, TX (US)

(73) Assignee: William Marsh Rice University, Houston, TX (US)

(21) Appl. No.: 17/779,689

(22) PCT Filed: Nov. 25, 2020
(86) PCT No.: PCT/US2020/062201

§ 371 Date: May 25, 2022

Related U.S. Application Data
(60) Provisional application No. 62/940,127, filed on Nov. 25, 2019.

(51) Int. Cl. C12Q 1/6869

(52) U.S. Cl. CPC C12Q 1/6869; C12Q 1/6806

(57) ABSTRACT

Provided herein are compositions and methods for assembling multiple DNA molecules into a linear concatemer, with applications to nanopore sequencing of DNA sequence variations.

### REFERENCE TO RELATED APPLICATIONS

The present application claims the priority benefit of U.S. provisional application No. 62/940,127, filed Nov. 25, 2019, the entire contents of which is incorporated herein by reference.

### STATEMENT REGARDING FEDERALLY SPONSORED RESEARCH

This invention was made with government support under Grant Nos. R01CA203964 and R01HG008752 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Oct. 27, 2020, is named RICEP0072WO_ST25.txt and is 14.3 kilobytes in size.

### BACKGROUND

### 1. Field

The present invention relates generally to the field of molecular biology. More particularly, it concerns compositions and methods for assembling multiple DNA molecules into a linear concatemer.

### 2. Description of Related Art

Nanopore sequencing (NS) is a method of sequencing where ionic current is passed through a nanopore and DNA sequence is decoded from the change in current as the nucleotides in the DNA molecule pass through the nanopore. There are a number of advantages to NS over short-read Next Generation Sequencing (NGS) like Illumina sequencing. NS allows long fragments of DNA, typically in 10-50 kb range to be sequenced, while NGS is limited to 150-300nt. The sequencing time is greatly reduced compared to Next Generation Sequencing (<1 hr for NS, compared to >24 hours to >72 hours for NGS) and sequencing data can be obtained in real time. Additionally, nanopore sequencing devices by Oxford Nanopore Technologies are small (approx. 10 cm×3 cm×3 cm) and no capital costs are required. A major disadvantage of NS over NGS is the higher intrinsic error rate (>7%) compared to 0.2% for Illumina NGS. The higher error rate prevents use of NS for directly detecting single nucleotide variations at low variant allele fraction (VAF).

In principle, ultradeep NS followed by background subtraction could allow detection of mutations, including single-nucleotide variants. However, the number of NS reads is limited, and NS of short DNA fragments or amplicons by NS results in lower throughput and lower quality reads due to higher error rates near the ends of DNA. Therefore, it is desirable to assemble short amplicons into longer DNA for NS sequencing.

Short DNA can be assembled by blunt end ligation. However, blunt end ligation is inefficient compared to cohesive end ligation, which makes it difficult to assemble long fragments from short 100-300 bp amplicons. Gibson assembly uses the sequential action of three enzymes, an exonuclease, a polymerase, and a ligase, to assemble DNA. The presence of exonuclease can lead to loss of sequence information and the polymerase can introduce errors in the sequence. The requirement for coordinated action of three enzymes also makes the system less robust and less efficient for long assemblies. As such, new methods are needed to assemble short DNA into long fragments for NS sequencing.

### SUMMARY

Provided herein are compositions and methods for assembling short DNA into long fragments by Linear DNA Assembly (LDA) using type IIS restriction enzyme digestion and ligation by DNA ligase. Type IIS restriction enzymes cut outside their recognition site, which allows assembly to occur by ligation even in the presence of the restriction enzyme. Provided are methods and reagents to improve assembly length by LDA. Also provided are methods that combine variant enrichment (e.g., Blocker Displacement Amplification (BDA)) with LDA for low VAF (0.1%) detection on NS platform, which is a 200-fold improvement over the current VAF detection capability of NS.

In one embodiment, provided herein are aqueous solutions for DNA monomer assembly, the solution comprising: a plurality of double-stranded DNA monomer species, each monomer species comprising, from 5′ to 3′: a type IIS restriction site in the (+) orientation (S1), a designed Left sticky end DNA sequence (1), an insert sequence (A), a second designed Right sticky end DNA sequence (1*), and a type IIS restriction site in the (−) orientation (S1*), wherein at least two different DNA monomers comprise the same Left sticky end DNA sequence, wherein at least two different DNA monomers comprise the same Right sticky end DNA sequence, and wherein the Left sticky end DNA sequence and the Right sticky end DNA sequence are complementary to and can form Watson-Crick base pairs with each other; a type IIS DNA restriction enzyme; a DNA ligase enzyme; and a chemical buffer suitable for the enzymatic functions of the type IIS DNA restriction enzyme and the DNA ligase enzyme.

In some aspects, the solutions further comprise a partially double-stranded

DNA seed molecule, the seed molecule comprising, from 5′ to 3′: a single-stranded Left sticky end DNA sequence (1); and a double stranded DNA region devoid of a type IIS restriction site (C). In some aspects, the solutions further comprise a partially double-stranded DNA seed molecule, the seed molecules comprising, from 5′ to 3′: a Left sticky end DNA sequence (1); a double stranded DNA region devoid of a type IIS restriction site (C); and a

Left sticky end DNA sequence (1).

In some aspects, the chemical buffer comprises between 20 mM and 150 mM Tris-HCl, between 2 mM and 50 mM MgCl2, between 0 mM and 50 mM DTT, and between 0.1 mM and 10 mM ATP, wherein the buffer exhibits a pH between 5.5 and 9.5 at 25° C. In some aspects, the chemical buffer comprises Tris-HCl at a concentration between 50 mM and 150 mM, between 75 mM and 150 mM, between 100 mM and 150 mM, between 20 mM and 125 mM, between 20 mM and 100 mM, between 20 mM and 75 mM, between 20 mM and 50 mM, or any range derivable therein. In some aspects, the chemical buffer comprises Tris-HCl at a concentration of about 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM 115 mM, 120 mM, 125 mM, 130 mM, 135 mM, 140 mM, 145 mM, or 150 mM. In some aspects, the chemical buffer comprises MgCl2 at a concentration between 2 mM and 50 mM, 5 mM and 50 mM, 10 mM and 50 mM, 15 mM and 50 mM, 20 mM and 50 mM, 25 mM and 50 mM, 30 mM and 50 mM, 2 mM and 45 mM, 2 mM and 40 mM, 2 mM and 35 mM, 2 mM and 30 mM, 2 mM and 25 mM, 10 mM and 40 mM, or any range derivable therein. In some aspects, the chemical buffer comprises MgCl2 at a concentration of about 2 mM, 5 mM, 10 mM, 15 mM, 20 mM, 25 mM, 30 mM, 35 mM, 40 mM, 45 mM, or 50 mM. In some aspects, the chemical buffer comprises DTT at a concentration of between 5 mM and 50 mM, between 10 mM and 50 mM, between 15 mM and 50 mM, between 20 mM and 50 mM, between 5 mM and 40 mM, between 2 mM and 25 mM, a range derivable therein any of the foregoing ranges, less than 45 mM, less than 40 mM, less than 35 mM, less than 30 mM, less than 25 mM, less than 20 mM, less than 15 mM, less than 10 mM, or less than 4 mM. In some aspects, the chemical buffer comprises DTT at a concentration of about 0 mM, 1 mM, 5 mM, 10 mM, 15 mM, 20 mM, 25 mM, 30 mM, 35 mM, 40 mM, 45 mM, or 50 mM. In some aspects, the chemical buffer comprises ATP at a concentration of between 0.1 mM and 9 mM, 0.1 mM and 8 mM, 0.1 mM and 7 mM, 0.1 mM and 6 mM, 0.1 mM and 5 mM, 1 mM and 10 mM, 2 mM and 9 mM, 3 mM and 8 mM, or any range derivable therein. In some aspects, the chemical buffer comprises ATP at a concentration of about 0.1 mM, 1 mM, 2 mM, 3 mM, 4 mM, 5 mM, 6 mM, 7 mM, 8 mM, 9 mM, or 10 mM. In some aspects, the chemical buffer exhibits a pH at 25° C. between 5.5 and 9.5, between 6 and 9.5, between 6.5 and 9.5, between 7 and 9.5, between 7.5 and 9.5, between 8 and 9.5, between 6 and 8, or any range derivable therein. In some aspects, the chemical buffer exhibits a pH at 25° C. of about 5.5, 6, 6.5, 7, 7.5, 8, 8.5, 9, or 9.5.

In some aspects, the type IIS DNA restriction enzyme is selected from BsaI, BbsI, BsmBI, BtgZI, Esp3I, and SapI. In some aspects, the S1 and S1* restriction sites correspond to the recognition site of the type IIS DNA restriction enzyme selected. In some aspects, the concentration of the type IIS DNA restriction enzyme is between 0.15 U/μL and 15 U/μL, between 0.25 U/μL and 15 U/μL, between 0.5 U/μL and 15 U/μL, between 1 U/μL and 15 U/μL, between 2 U/μL and 15 U/μL, between 5 U/μL and 15 U/μL, between 0.15 U/μL and 10 U/μL, between 1 U/μL and 10 U/μL, or any range derivable therein. In some aspects, the concentration of the type IIS DNA restriction enzyme is about 0.15, 0.2, 0.25, 0.5, 0.75, 1, 1.5, 2, 3, 4, 5, 6, 7, 8, 9, 10, 11, 12, 13, 14, or 15 IR

In some aspects, the DNA ligase enzyme is selected from T4 DNA ligase, T7 DNA ligase, T3 DNA ligase, Taq DNA ligase, and E. Coli DNA ligase. In some aspects, the concentration of the DNA ligase is between 5 U/μL and 500 U/μL, between 5 U/μL and 400 U/μL, between 5 U/μL and 300 U/μL, between 5 U/μL and 200 U/μL, between 5 U/μL and 100 U/μL, between 5 U/μL and 50 U/μL, between 50 U/μL and 500 U/μL, between 100U/μL and 500 U/μL, between 50 U/μL and 300 U/μL, between 50 U/μL and 200 U/μL, or any range derivable therein. In some aspects, the concentration of the DNA ligase is about 5, 10, 15, 20, 25, 30, 40, 50, 60, 70, 80, 90, 100, 150, 200, 250, 300, 350, 400, 450, or 500 U/μL.

In some aspects, the Left sticky end DNA sequence and the Right sticky end DNA sequence each have a length of 2-6 nucleotides (e.g., having a length of 2, 3, 4, 5, 6, or 7 nucleotides). In some aspects, the insert sequence of each monomer has a length between 40 nt and 2,000 nt, between 100 nt and 2,000 nt, between 500 nt and 2,000 nt, between 40 nt and 1,000 nt, between 40 nt and 500 nt, between 40 nt and 100 nt, or any range derivable therein. In some aspects, the insert sequence of each monomer has a length of about 40 nt, 50 nt, 60 nt, 70 nt, 80 nt, 90 nt, 100 nt, 150 nt, 200 nt, 250 nt, 300 nt, 400 nt, 500 nt, 750 nt, 1,000 nt, 1,250 nt, 1,500 nt, 1,750 nt, or 2,000 nt.

In some aspects, the total concentration of all DNA monomers is between 5 nM and 5 μM, 5 nM and 1 μM, 5 nM and 500 nM, 5 nM and 100 nM, 100 nM and 5 500 nM and 5 100 nM and 1 100 nM and 5 or any range derivable therein. In some aspects, the total concentration of all DNA monomers is about 5 nM, 10 nM, 20 nM, 50 nM, 100 nM, 200 nM, 500 nM, 1 μM, 2 μM, 3 μM, 4 μM, or 5 μM.

In some aspects, the total concentration of all DNA monomers is 1x to 1000x (e.g., 1x to 100x, 1x to 50 x, 10x to 1000x, 50x to 1000x, 100x to 1000x, 100x to 500x, or any range derivable therein) the concentration of partially double-stranded DNA seed molecules. In some aspects, the total concentration of all DNA monomers is 1x, 2x, 5x, 10x, 25x, 50x, 100x, 200x, 500x, or 1000x the concentration of partially double-stranded DNA seed molecules.

In one embodiment, provided herein are methods for linear assembly of DNA concatemers from a plurality of double-stranded DNA monomers, each monomer species comprising, from 5′ to 3′: a type IIS restriction site in the (+) orientation (S1), a designed Left sticky end DNA sequence (1), an insert sequence (A), a second designed Right sticky end DNA sequence (1*), and a type IIS restriction site in the (−) orientation (S1*); wherein at least two different DNA monomers comprise the same Left sticky end DNA sequence, wherein at least two different DNA monomers comprise the same Right sticky end DNA sequence, and wherein the Left sticky end DNA sequence and the Right sticky end DNA sequence are complementary to and can form Watson-Crick base pairs with each other; the method comprising: mixing the DNA monomers with a type IIS DNA restriction enzyme, a DNA ligase enzyme, and a chemical buffer suitable for the enzymatic functions of the type IIS DNA restriction enzyme and the DNA ligase enzyme; and thermal cycling the solution between 5 cycles and 100 cycles (e.g., 5-50 cycles, 10-50 cycles, 5-40 cycles, 10-40 cycles, or any range derivable therein), with each cycle comprising between 5 seconds and 5 minutes (e.g., 5-60 seconds, 5-120 seconds, 30-60 seconds, 30-120 seconds, 20-60 seconds, 20-120 seconds, or any range derivable therein) at a temperature between 30° C. and 45° C. (e.g., at 30, 31, 32, 33, 34, 35, 36, 37, 38, 39, 40, 41, 42, 43, 44, or 45° C., or any range derivable therein), and between 30 seconds and 30 minutes (e.g., 30-1000 seconds, 60-1000 seconds, 30-1500 seconds, 60-1500 seconds, 30-500 seconds, 60-200 seconds, 30-200 seconds, 60-500 seconds, 30-250 seconds, 60-250 seconds, or any range derivable therein) at a temperature between 10° C. and 25° C. (e.g., at 10, 11, 12, 13, 14, 15, 16, 17, 18, 19, 20, 21, 22, 23, 24, or 25° C., or any range derivable therein).

In some aspects, a partially double-stranded DNA seed molecule is mixed with the monomer molecules before thermal cycling, the seed molecule comprising, from 5′ to 3′: a single-stranded Left sticky end DNA sequence (1); and a double stranded DNA region devoid of a type IIS restriction site (C). In some aspects, a partially double-stranded DNA seed molecule is mixed with the monomer molecules before thermal cycling, the seed molecule comprising, from 5′ to 3′: a Left sticky end DNA sequence (1); a double stranded DNA region devoid of a type IIS restriction site (C); and a Left sticky end DNA sequence (1). In some aspects, a partially double-stranded DNA seed molecule is mixed with the monomer molecules before thermal cycling, the seed molecule comprising, from 5′ to 3′: a Left sticky end DNA sequence (1); a double stranded DNA region devoid of a type IIS restriction site (C) and a unique barcode; and a sticky end DNA sequence (2) for appending adapters for nanopore sequencing.

In some aspects, the DNA monomers are generated by a method comprising:

amplifying a DNA template by multiplex polymerase chain reaction (PCR) amplification, comprising: adding to a DNA template solution (1) a set of forward DNA primers comprising, from 5′ to 3′: a type IIS restriction site in the (+) orientation (S1), a designed Left sticky end DNA sequence (1), and a gene-specific sequence; (2) a set of reverse DNA primers comprising, from 5′ to 3′: a type IIS restriction site in the (+) orientation (S1), a designed Right sticky end DNA sequence (1*), and a gene-specific sequence; (3) a DNA polymerase; and (4) a chemical buffer suitable for PCR amplification; thermal cycling the solution between 5 cycles and 60 cycles (e.g., 5-60 cycles, 10-60 cycles, 5-50 cycles, 10-50 cycles, 5-40 cycles, 10-40 cycles, or any range derivable therein), with each cycle comprising between 5 seconds and 1 minute (e.g., 5-60 seconds, 5-50 seconds, 30-60 seconds, 30-50 seconds, 20-60 seconds, 20-50 seconds, or any range derivable therein) at a temperature between 90° C. and 100° C. (e.g., at 90, 91, 92, 93, 94, 95, 96, 97, 98, 99, or 100° C., or any range derivable therein), and between 30 seconds and 2 minutes (e.g., 30-60 seconds, 60-120 seconds, 40-60 seconds, 40-120 seconds, or any range derivable therein) at a temperature between 55° C. and 72° C. (e.g., at 55, 56, 57, 58, 59, 60, 61, 62, 63, 64, 65, 66, 67, 68, 69, 70, 71, or 72° C., or any range derivable therein).

In some aspects, a set of gene-specific DNA Blockers are additionally added to the DNA template solution. In some aspects, the region of the DNA template that the Blockers bind overlaps with that of the forward DNA primers by between 4 and 15 nucleotides (e.g., 4, 5, 6, 7, 8, 9, 10, 11, 12, 13, 14, or 15 nucleotides, or any range derivable therein). In some aspects, the standard free energy of the forward primer displacing the Blocker at 60° C. in 5 mM Mg²⁺is between 0 kcal/mol and +5 kcal/mol (e.g., 0-4, 0-3, 0-2, 0-1, 1-5, 1-4, 1-3, 1-2, 2-5, 2-4, 2-3, 3-5, 3-4, 4-5 kcal/mol, or any range derivable therein).

In one embodiment, provided herein are methods of generating DNA monomers for linear assembly, the method comprising: obtaining a DNA sample solution that comprises a DNA template; amplifying the DNA template by multiplex polymerase chain reaction (PCR) amplification, comprising: adding to the DNA solution (1) a set of forward DNA primers comprising, from 5′ to 3′: a type IIS restriction site in the (+) orientation (S1), a designed Left sticky end DNA sequence (1), and a gene-specific sequence; (2) a set of reverse DNA primers comprising, from 5′ to 3′: a type IIS restriction site in the (+) orientation (S1), a designed Right sticky end DNA sequence (1*), and a gene-specific sequence; (3) a DNA polymerase; and (4) a chemical buffer suitable for PCR amplification; thermal cycling the solution between 5 cycles and 60 cycles(e.g., 5-60 cycles, 10-60 cycles, 5-50 cycles, 10-50 cycles, 5-40 cycles, 10-40 cycles, or any range derivable therein), with each cycle comprising between 5 seconds and 1 minute (e.g., 5-60 seconds, 5-50 seconds, 30-60 seconds, 30-50 seconds, 20-60 seconds, 20-50 seconds, or any range derivable therein) at a temperature between 90° C. and 100° C. (e.g., at 90, 91, 92, 93, 94, 95, 96, 97, 98, 99, or 100° C., or any range derivable therein), and between 30 seconds and 2 minutes (e.g., 30-60 seconds, 60-120 seconds, 40-60 seconds, 40-120 seconds, or any range derivable therein) at a temperature between 55° C. and 72° C. (e.g., at 55, 56, 57, 58, 59, 60, 61, 62, 63, 64, 65, 66, 67, 68, 69, 70, 71, or 72° C., or any range derivable therein). In some aspects, the forward and/or reverse primers further comprise a UMI barcode.

In some aspects, a set of gene-specific DNA Blockers are additionally added to the DNA template solution. In some aspects, the region of the DNA template that the Blockers bind overlaps with that of the forward DNA primers by between 4 and 15 nucleotides (e.g., 4, 5, 6, 7, 8, 9, 10, 11, 12, 13, 14, or 15 nucleotides, or any range derivable therein). In some aspects, the standard free energy of the forward primer displacing the Blocker at 60° C. in 5 mM Mg²⁺is between 0 kcal/mol and +5 kcal/mol (e.g., 0-4, 0-3, 0-2, 0-1, 1-5, 1-4, 1-3, 1-2, 2-5, 2-4, 2-3, 3-5, 3-4, 4-5 kcal/mol, or any range derivable therein).

In one embodiment, provided herein are methods for preparing a solution of heterogeneous DNA concatemers, the method comprising: preparing a set of DNA monomers from a DNA template sample according to the method of any one of the present embodiments; purifying the monomers to remove unreacted primers and enzymes; and performing linear DNA assembly according to the method of one of the present embodiments. In some aspects, purifying the monomers comprises using either an affinity column or magnetic beads.

In some aspects, a set of gene-specific DNA Blockers are additionally added to the DNA template solution. In some aspects, the region of the DNA template that the Blockers bind overlaps with that of the forward DNA primers by between 4 and 15 nucleotides (e.g., 4, 5, 6, 7, 8, 9, 10, 11, 12, 13, 14, or 15 nucleotides, or any range derivable therein). In some aspects, the standard free energy of the forward primer displacing the Blocker at 60° C. in 5 mM Mg²⁺is between 0 kcal/mol and +5 kcal/mol (e.g., 0-4, 0-3, 0-2, 0-1, 1-5, 1-4, 1-3, 1-2, 2-5, 2-4, 2-3, 3-5, 3-4, 4-5 kcal/mol, or any range derivable therein).

In one embodiment, provided herein are methods for targeted nanopore sequencing of gene regions of interest, the method comprising: obtaining a DNA sample of interest comprising a DNA template; preparing a set of DNA monomers from the DNA template according to the method of any one of the present embodiments; purifying the monomers to remove unreacted primers and enzymes; performing linear DNA assembly according to the method of any one of the present embodiments; purifying the concatemers to remove unreacted monomers, Type IIS reaction side products, and enzymes; appending adapters for nanopore sequencing to the purified concatemers; purifying the adapter-appended concatemers to remove excess adapters and enzymes; and performing nanopore sequencing.

In one embodiment, provided herein are methods for constructing a monomer species comprising, from 5′ to 3′: a type IIS restriction site in the (+) orientation (S1), a designed Left sticky end DNA sequence (1), an insert sequence (A), a second designed Right sticky end DNA sequence (1*), and a type IIS restriction site in the (−) orientation (S1*); wherein at least two different DNA monomers comprise the same Left sticky end DNA sequence, wherein at least two different DNA monomers comprise the same Right sticky end DNA sequence, and wherein the Left sticky end DNA sequence and the Right sticky end DNA sequence are complementary to and can form Watson-Crick base pairs with each other; the method comprising: obtaining a solution of double-stranded DNA inserts of interest; performing a first ligation reaction on a first portion of the solution with a double stranded DNA adaptor comprising: a type IIS restriction site in the (+) orientation (S1), and a designed Left sticky end DNA sequence (1); performing a second reaction ligation reaction on a second portion of the solution with a double stranded DNA adaptor comprising: a type IIS restriction site in the (+) orientation (S1), and a designed Right sticky end DNA sequence (1*); and mixing the products of the first and second ligations reactions in a solution in a chemical buffer conducive to ligation. In some aspects, the double-stranded DNA inserts are dA-tailed prior to performing the ligation.

In one embodiment, provided herein are methods for targeted nanopore sequencing of gene regions of interest, the method comprising: obtaining a DNA sample of interest comprising a DNA template; preparing a set of DNA monomers from the DNA template according to the method of one of the present embodiments; purifying the monomers to remove unreacted primers and enzymes; performing linear DNA assembly according to the method of any one of the present embodiments; purifying the concatemers to remove unreacted monomers, Type IIS reaction side products, and enzymes; appending adapters for nanopore sequencing to the purified concatemers; purifying the adapter-appended concatemers to remove excess adapters and enzymes; and performing nanopore sequencing.

In some aspects, the step of mixing the DNA monomers further comprises mixing with two single-stranded destructive probes, the first single-stranded destructive probe comprising, from 5′ to 3′, a type IIS recognition sequence (S1), and a Left sticky end DNA sequence (1); and the second single-stranded destructive probe comprising, from 5′ to 3′: a type IIS recognition sequence (S1), and the Right sticky end DNA sequence (1*). In some aspects, the concentration of the destructive probe is between 1x and 100x of the total concentration of the DNA monomers. In some aspects, the destructive probes have chemical modifications that prevents restriction digestion. In some aspects, the modifications are selected from phosphorothioate-substituted backbone, sugar modified nucleotides (e.g., 2′Fluoro, 2′-OMe), inverted DNA nucleotides, methylated bases, DNA with carbon spacers, or DNA with polyethylene glycol (PEG) spacers.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Schematic representation of the mechanism of Linear DNA Assembly (LDA). FIG. 1A. Homo-polymer assembly using LDA. Monomers of different family (A and B) have orthogonal sticky ends. Family A monomers have sticky ends 1 and 1* that are complementary to each other, while family B monomers have sticky ends 2 and 2* that are complementary to each other. Assembly reaction is carried out by cycling between a restriction step at 37° C. for 30 s to 2 min and a ligation step at 16° C. for 2 min to 10 min. Hybridization between family A and B monomers does not occur during the ligation step leading to assembly of homo-polymers containing monomers from the same family. FIG. 1B.

Hetero-polymer assembly using LDA. Monomers of different family (A and B) have the same sticky ends 1 and 1*. Hybridization between family A and B monomers can occur during the ligation step leading to assembly of hetero-polymers containing monomers from different families. S1 is the type IIS restriction enzyme recognition site in the (+) orientation and S1* is the recognition site in the (−) orientation.

FIG. 2. Histogram showing length distribution of DNA assembled by LDA. 182 bp DNA monomers were assembled by LDA. No size selection was performed. Length distribution was analyzed by NS.

FIGS. 3A-3B. Preparation of DNA monomers for LDA. FIG. 3A.

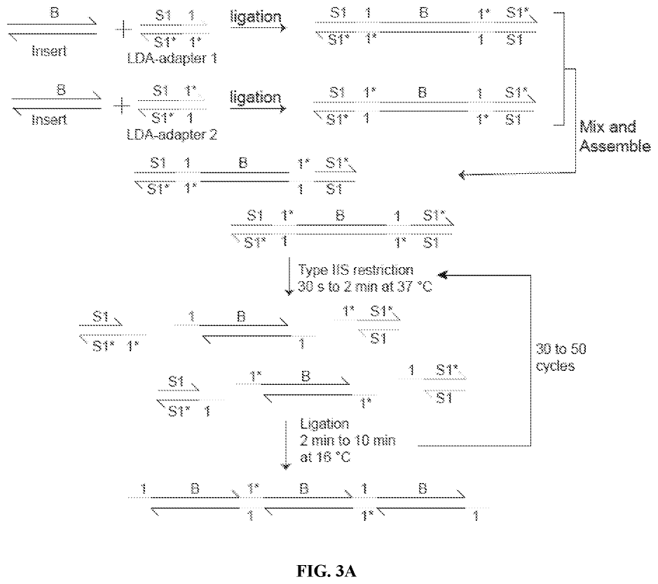

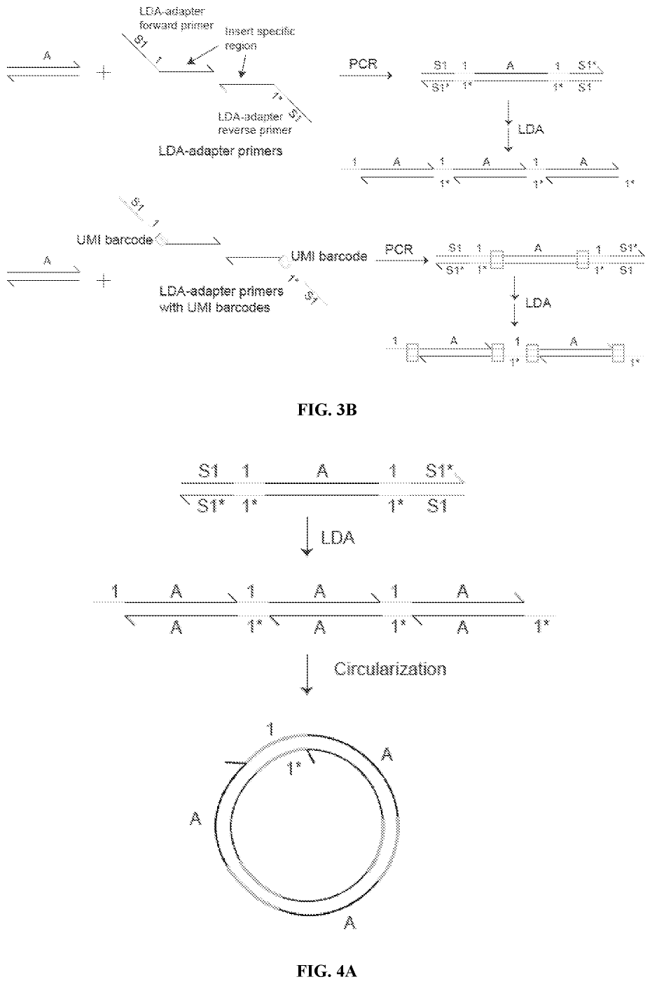

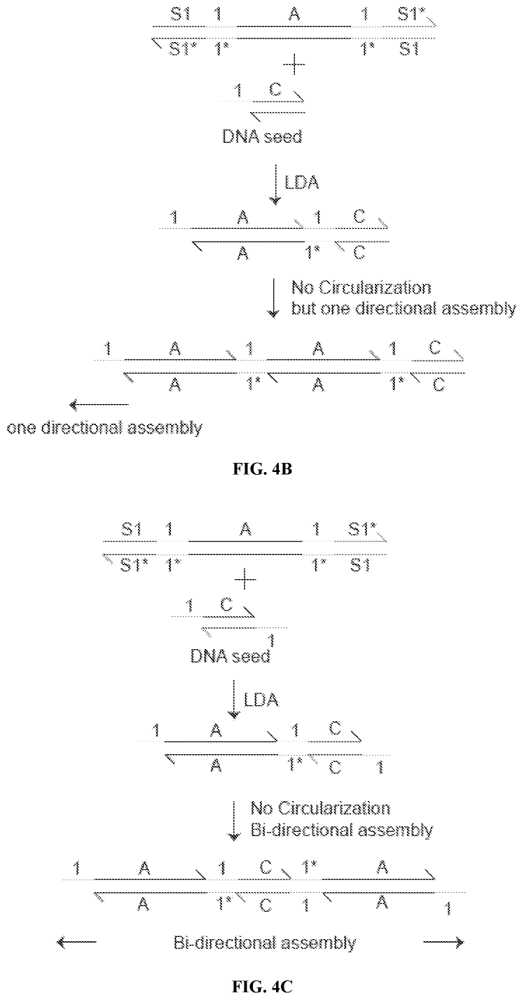

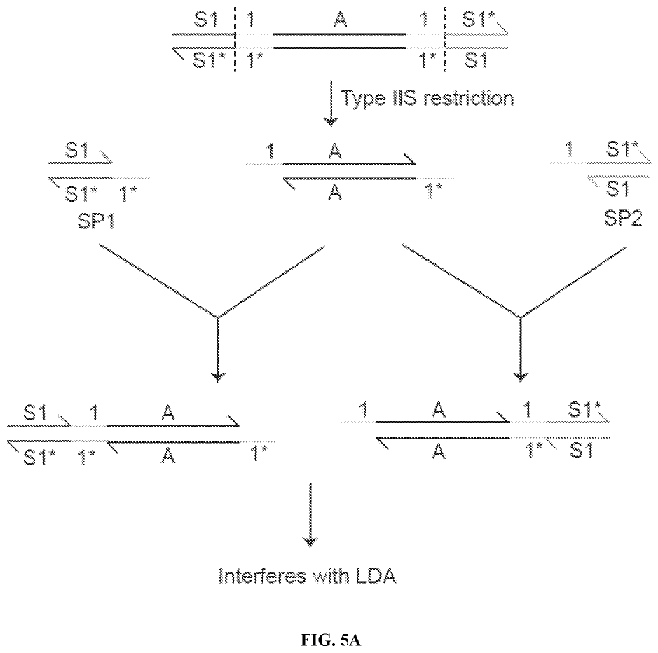

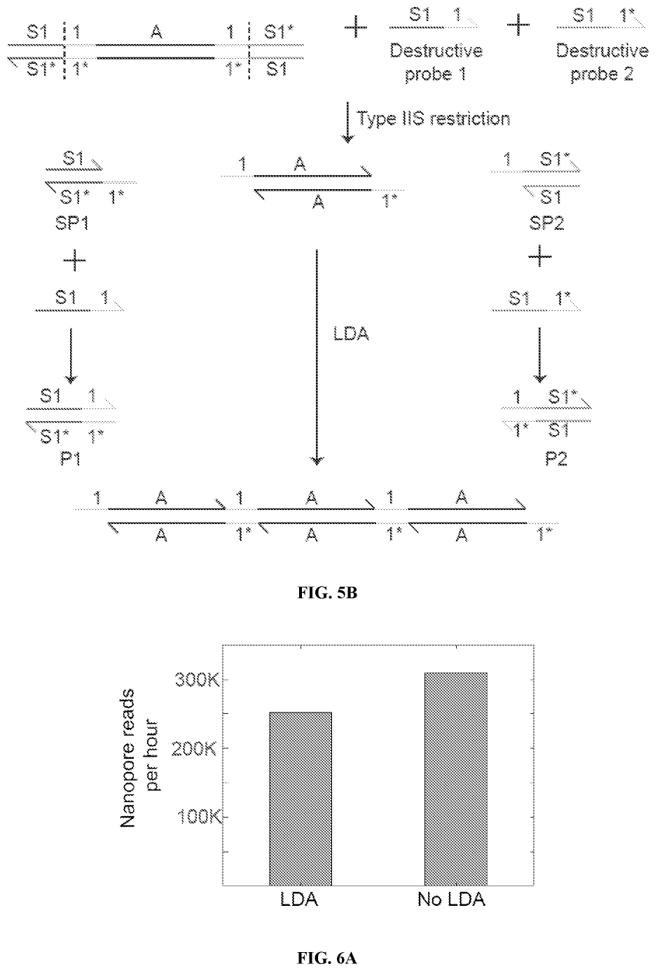

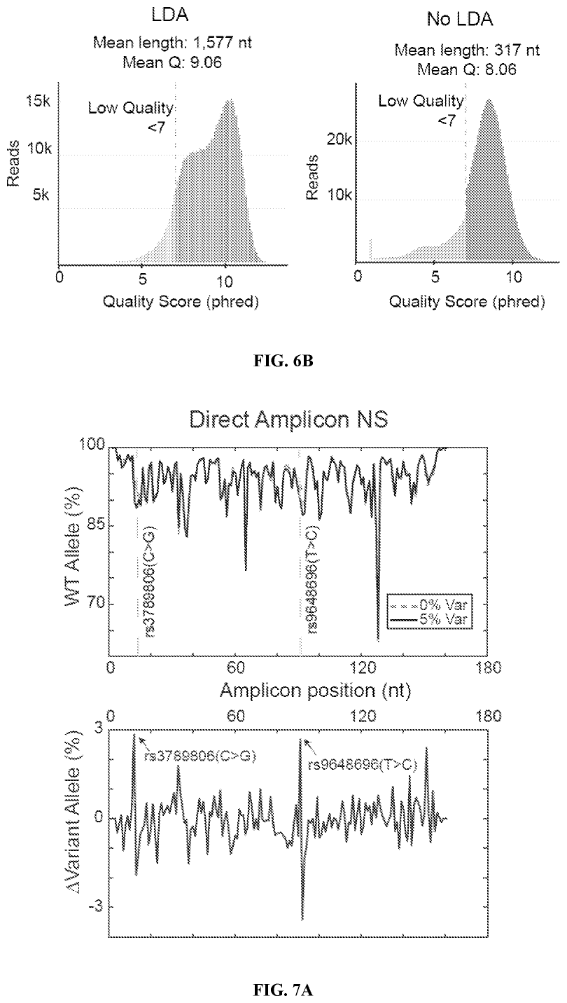

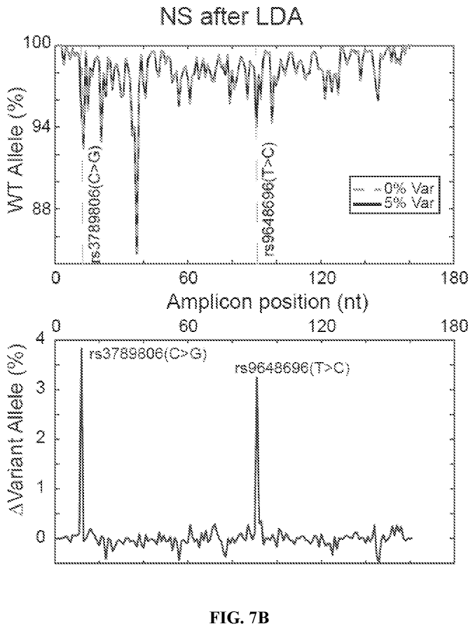

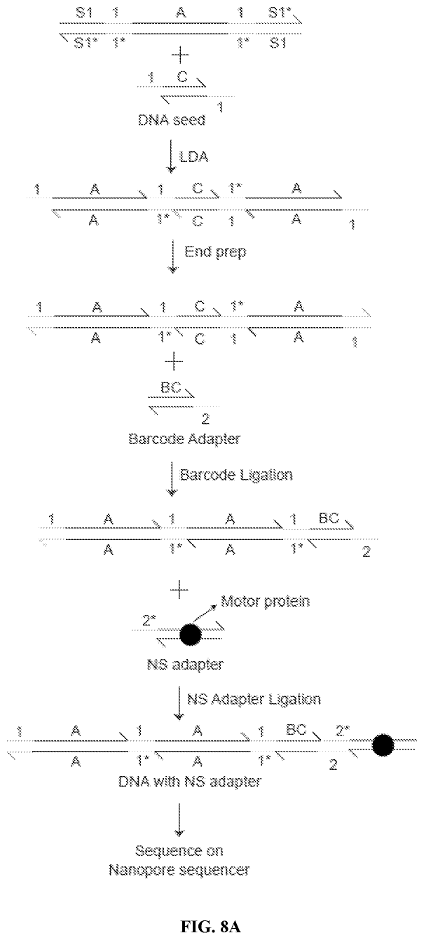

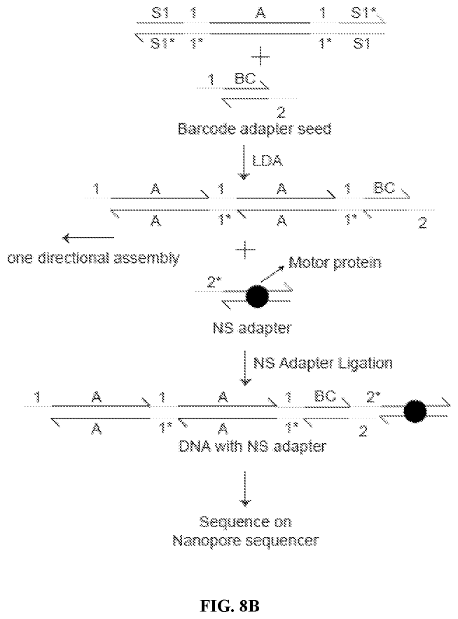

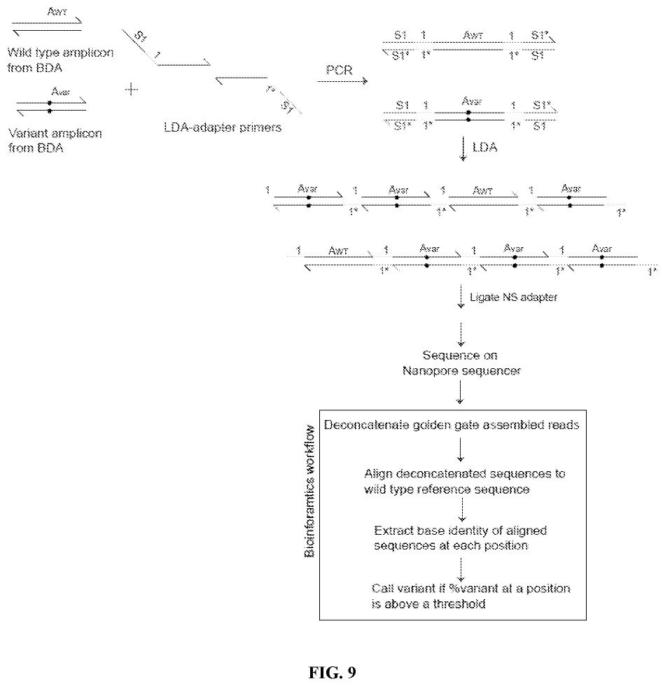

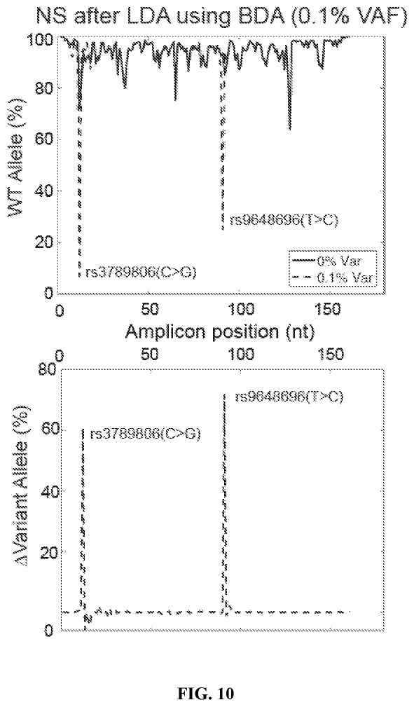

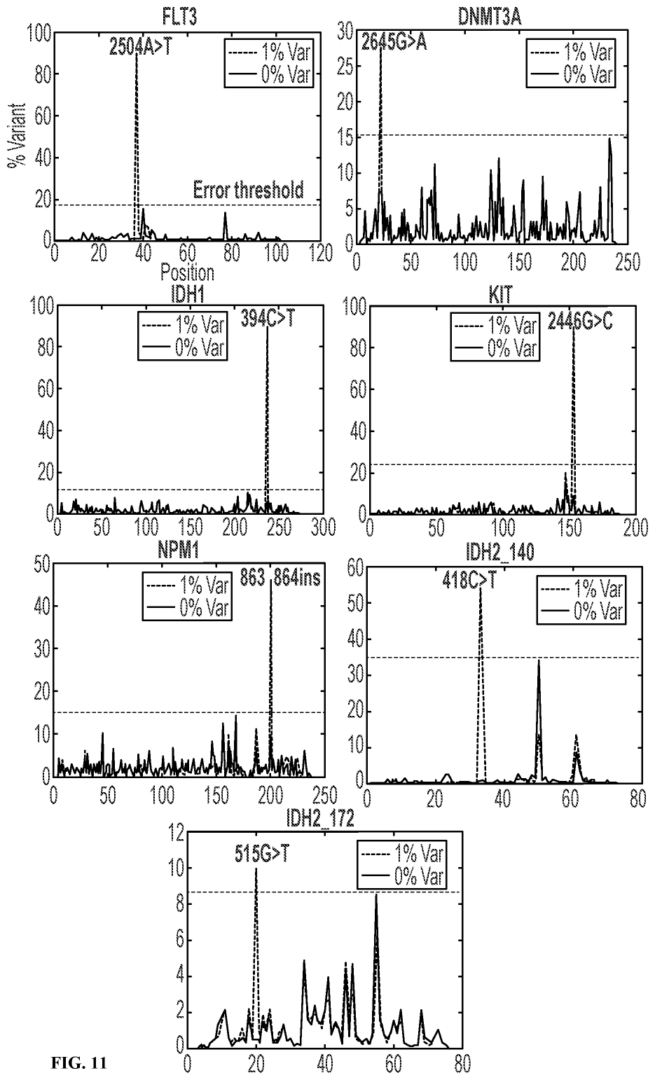

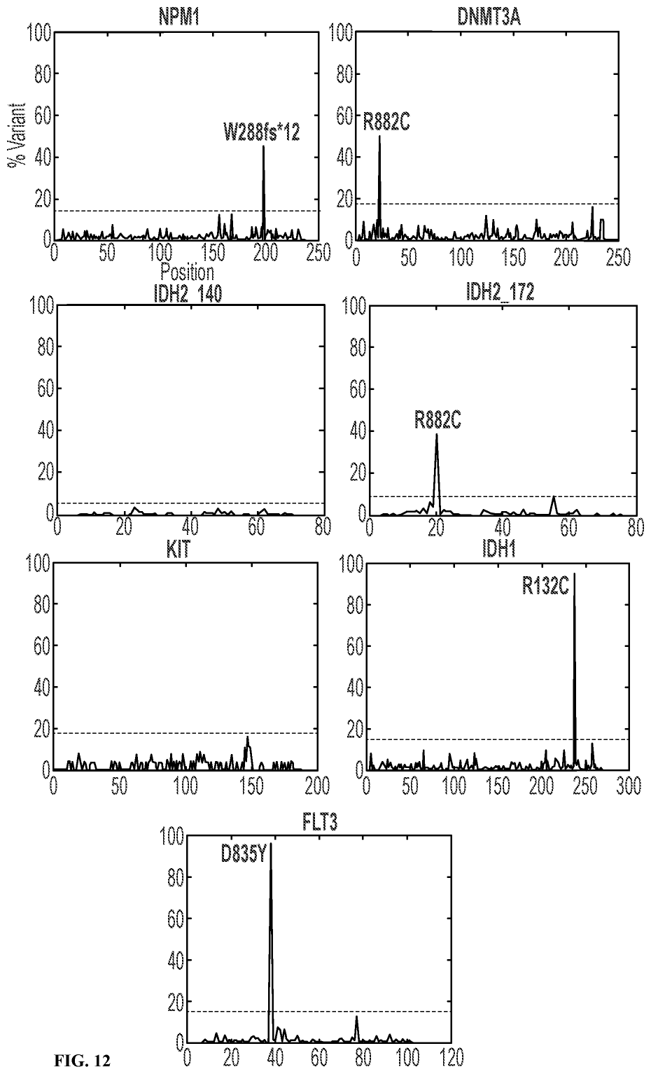

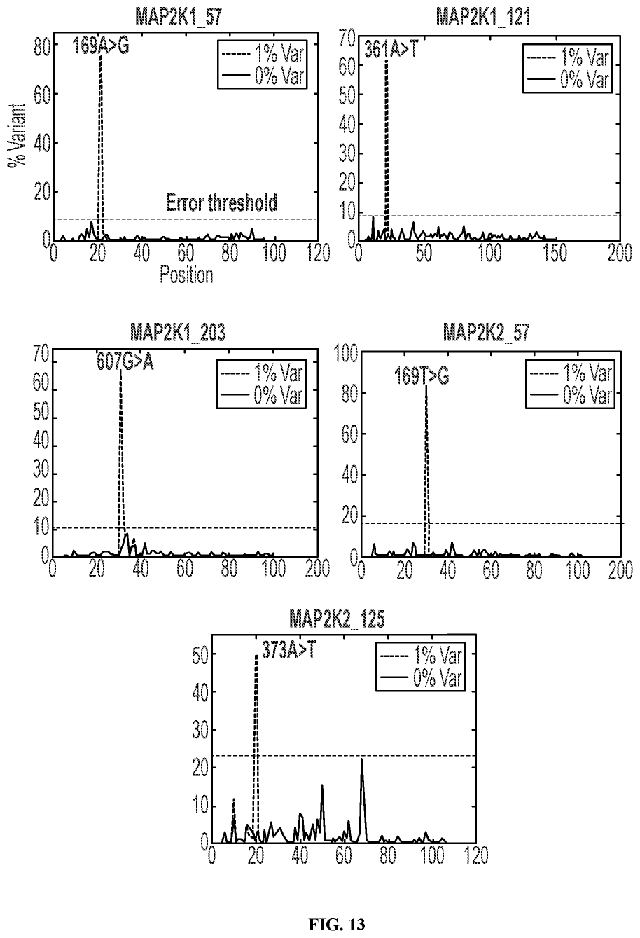

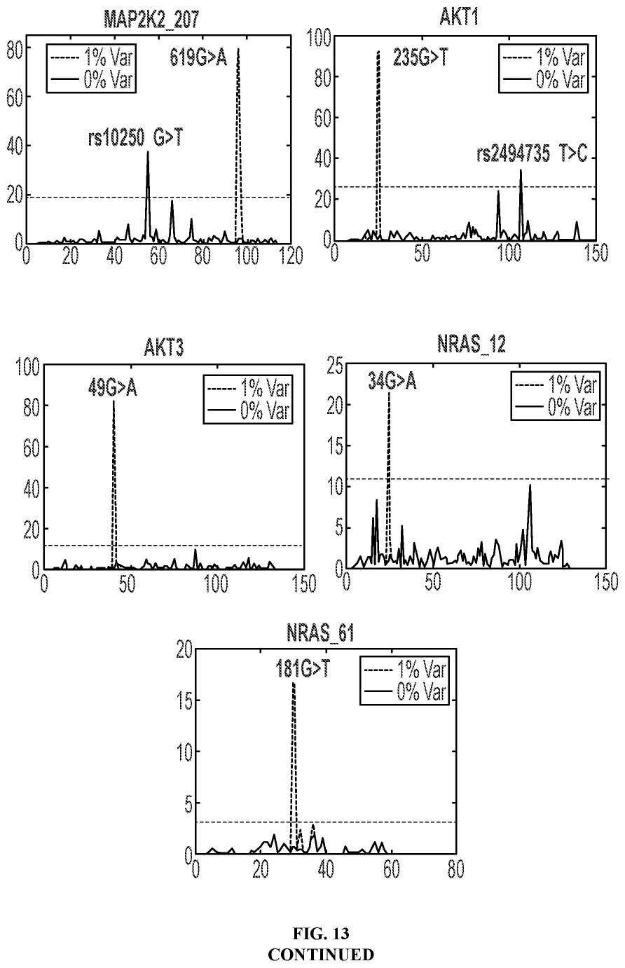

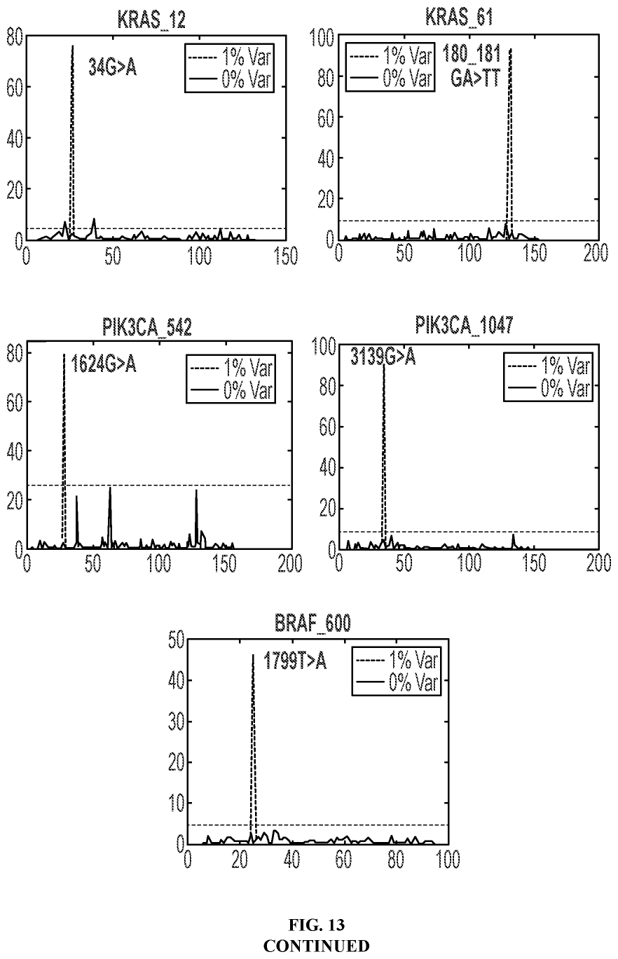

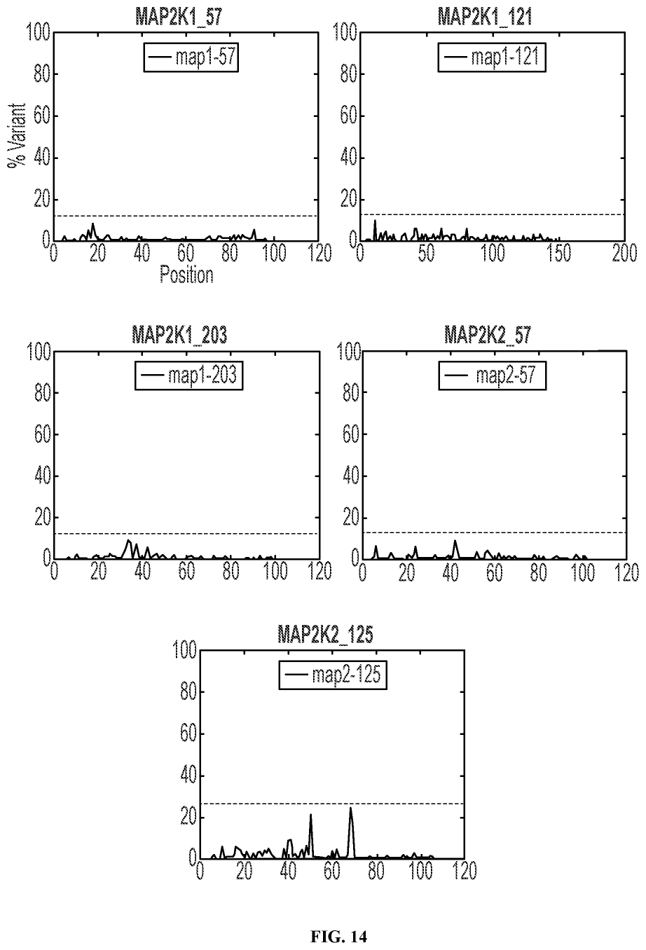

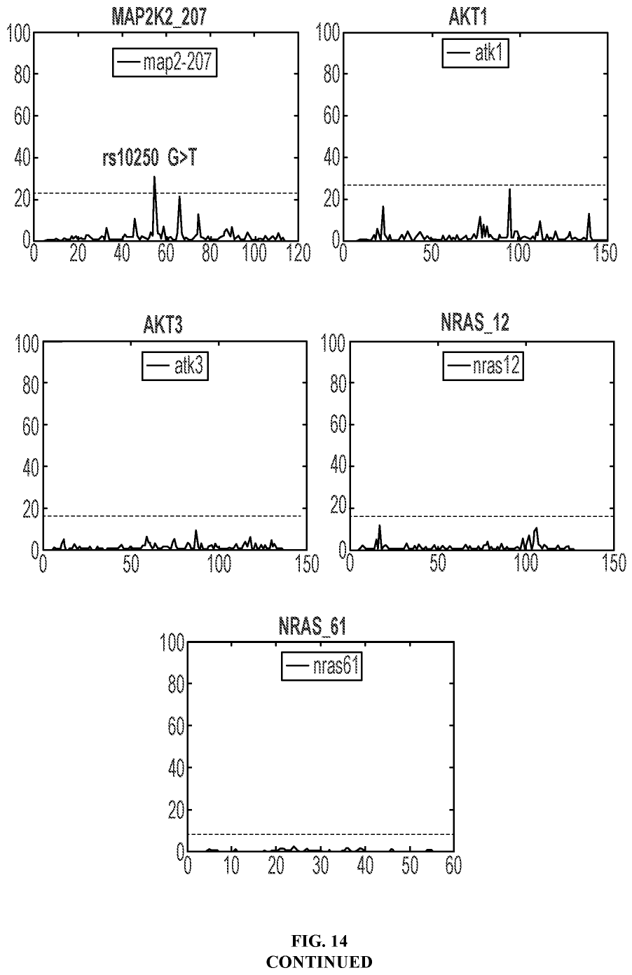

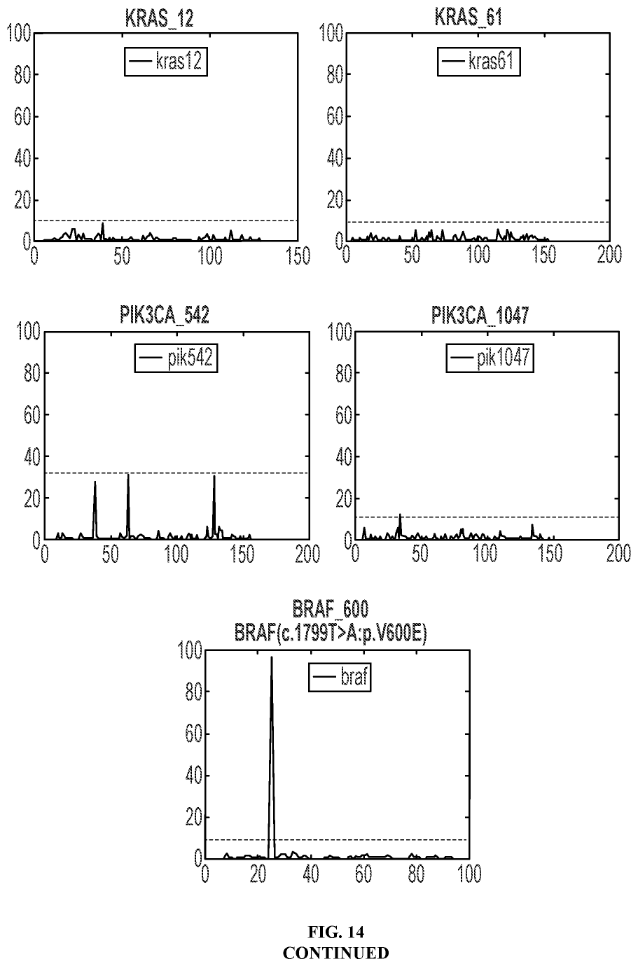

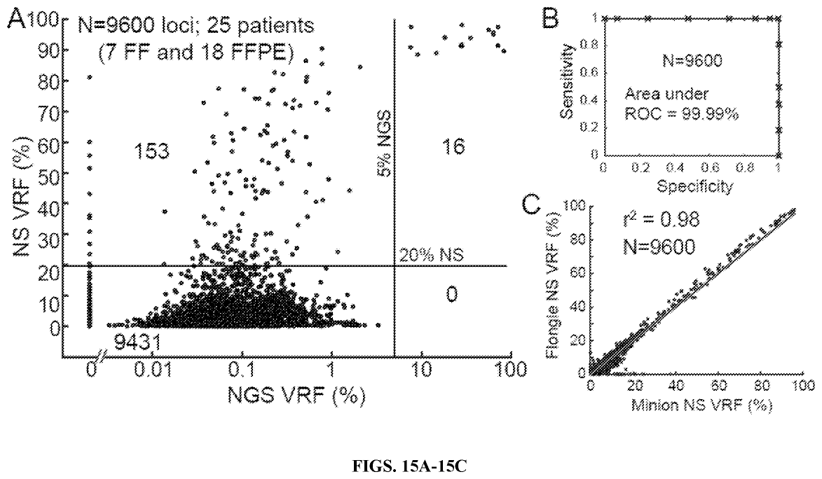

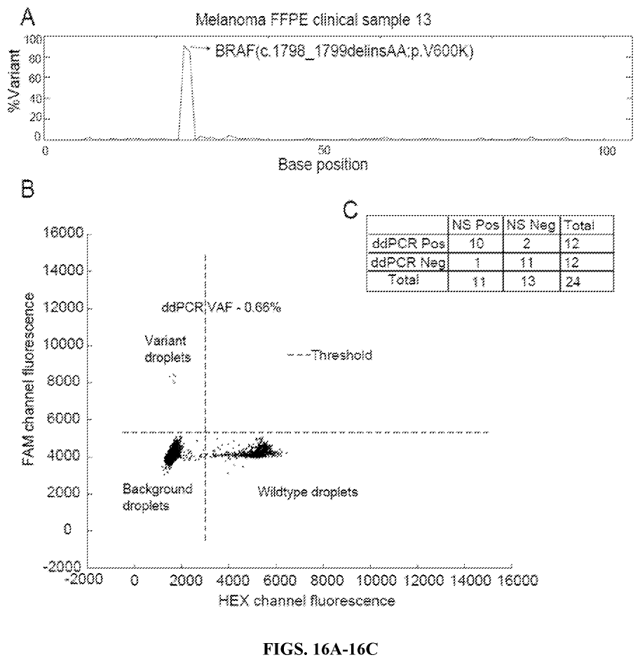

Preparation of DNA monomers by dA-tailing and ligation of LDA-adapters. LDA-adapterl and LDA-adpater2 are ligated to dA-tailed insert DNA in two separate ligation reactions. The ligated monomers from the two reactions are mixed and included in the LDA reaction. Monomers with LDA-adapter1 have sticky end 1, while monomers with LDA-adapter2 have sticky end 1* after the restriction step. These two monomer populations can ligate with each other during the ligation step to form linear assemblies. FIG. 3B. Preparation of DNA monomers by PCR. LDA-adapter forward primer contains type IIS restriction enzyme recognition site in the (+) orientation (S1) followed by the Left sticky end sequence (1) followed by insert specific sequence. LDA-adapter reverse primer contains type IIS restriction enzyme recognition site in the (−) orientation (S1*) followed by the Right sticky end sequence (1*) followed by insert specific sequence. LDA-adapter primers can also contain UMI barcodes. PCR of an insert DNA with LDA-adapter primers generates amplicons that can be used as monomers for LDA.

FIGS. 4A-4C. Directional assembly of monomers on a seed. FIG. 4A. Long linear assemblies formed during LDA can circularize by self-hybridization during the ligation step. FIG. 4B. One directional assembly of monomers on a seed. Mixing a DNA seed with one sticky end sequence at low concentration during LDA will lead to one directional assembly of monomers on the seed. Long linear assemblies on the seed will not circularize, due to lack of complementary sticky ends. FIG. 4C. Bi-directional assembly of monomers on a seed. Mixing a DNA seed with two sticky end sequences at low concentration during LDA will lead to bi-directional assembly of monomers on the seed. Long linear assemblies on the seed will not circularize, due to lack of complementary sticky ends.

FIGS. 5A-5B. Blocking side product assembly. FIG. 5A. Ligation of type IIS restriction side product into a growing linear assembly can terminate assembly at that end until another restriction event removes the side product from the assembly. FIG. 5B. Blocking side product assembly by use of destructive probes. Destructive probes 1 and 2 can each react with side products SP1 and SP2 respectively and block ligation of the side product into a growing assembly.

FIGS. 6A-6B. LDA improves NS read throughput and quality. FIG. 6A.

NS read throughput for DNA assembled by LDA (mean size of 1577 nt) is comparable to throughput of DNA monomers without assembly (mean size of 317 nt). FIG. 6B. The average read quality is higher for DNA assembled by LDA compared to DNA monomers without assembly.

FIGS. 7A-7B. Variant allele detection by NS. FIG. 7A. Results for 5% variant allele detection by NS of 161 nt PCR amplicon without LDA. The amplicon was designed to cover 2 SNPs, rs3789806(C>G) and rs9648696(T>C). The 0% variant sample is NA18537 human genomic DNA, and the 5% variant sample is a mixture containing 95% NA18537 and 5% NA18562. The top panel shows the fraction of reads at each nucleotide position corresponding to the wildtype (NA18537 homozygous) allele. Note that due to NS intrinsic error the WT allele percentage at four positions is less than 85%. The bottom panel shows the ΔVariant allele %, which is the fraction of reads mapped to the highest frequency variant allele in the 5% variant sample, minus the variant allele frequency in the matched normal 0% variant sample. The ΔVariant allele % is noisy and based on these results, the 2SNPs at 5% VAF cannot be distinguished from the false positive signal at position 151nt. FIG. 7B. NS results of the same PCR amplicon as in (A) assembled by LDA, using similar number of reads. The higher quality and depth from LDA significantly reduce the stochastic noise and allows confident detection of 5% VAF in the bottom panel.

FIGS. 8A-8B. Short library preparation for NS by LDA. FIG. 8A. Normal library preparation for NS after LDA. Assembled DNA is end prepared to add phosphate groups to the 5′ end and dA to the 3′ end of DNA. The end prepared DNA is then ligated to a barcode adapter containing a unique barcode sequence (BC) and a sticky end sequence (2). The NS adapter containing motor protein and a compatible sticky end sequence (2*) to that of the barcode adapter (2) is then ligated to the DNA for sequencing. FIG. 8B. Shortened library preparation for NS using a barcode adapter seed containing a single stranded Left sticky end sequence (1), a double stranded DNA region devoid of type IIS restriction site but containing a unique barcode sequence for sample identification (BC) and a Right sticky end sequence (2) for NS adapter ligation. DNA assembled by LDA has the barcode ligated to one end, which can be ligated to the NS adapter directly without end preparation and barcode ligation steps of the normal NS library preparation workflow.

FIG. 9. Workflow for target sequencing of mutations using BDA, LDA and nanopore sequencing (NS). Blocker displacement amplification (BDA) enriches variant amplicons over wild-type amplicons from a sample containing low VAF. Amplicons from BDA are prepared as monomers for LDA by PCR using LDA-adapter primers. The amplicons from this PCR is used as monomers for LDA to make long linear assemblies of the BDA amplicons. The assembled DNA is then sequenced on the Minion using ligation sequencing library preparation method. The sequencing data is then analyzed using the bioinformatics workflow mentioned to call for variants.

FIG. 10. Detection of 0.1% VAF by NS after LDA using BDA. Two SNPs rs3789806(C>G) and rs9648696(T>C) present at 0.1% VAF are detected on NS by combining BDA with LDA. The 0% variant sample is human genomic DNA (gDNA)

NA18537 and 0.1% variant sample is 0.1% human gDNA NA18562 in NA18537. gDNA sample NA18562 bears the two SNPs that were detected. BDA probes were designed for the SNP rs3789806(C>G), while the SNP rs9648696(T>C) occurs in cis. In the top panel, fraction of reads at each nucleotide position corresponding to the wildtype (NA18537 homozygous) allele is plotted. The two SNPs can be clearly detected in the 0.1% variant sample. The bottom panel shows the ΔVariant allele %, which is the fraction of reads mapped to the highest frequency variant allele in the 0.1% variant sample, minus the variant allele frequency in the matched normal 0% variant sample.

FIG. 11. NS results from AML 7-plex panel on synthetic genes spiked at 1% VAF into human genomic DNA -NA18537 (1% Var sample) and 100% NA18537 (0% Var sample). For each amplicon, percentage of variant reads at each nucleotide position corresponding to the human reference genome sequence (hg38) is plotted. The mutations present in the synthetic genes at 1% VAF are detected above the error threshold for all 7 amplicons in the 1% Var sample but not in the 0% Var sample.

FIG. 12. NS results from AML 7-plex panel on human cancer cell-line (11D892) genomic DNA. For each amplicon, percentage of variant reads at each nucleotide position corresponding to the human reference genome sequence (hg38) is plotted. HD892 has mutations at 5% VAF in NPM1, DNMT3A, IDH1, IDH2 172 and FLT3, all of which are detected above the error threshold.

FIG. 13. NS results from melanoma 15-plex panel on synthetic genes spiked at 1% VAF into human genomic DNA -NA18562 (1% Var sample) and 100% NA18562 (0% Var sample). For each amplicon, percentage of variant reads at each nucleotide position corresponding to the human reference genome sequence (hg38) is plotted. The mutations present in the synthetic genes at 1% VAF are detected above the error threshold for all 15 amplicons in the 1% Var sample but not in the 0% Var sample. Non-pathogenic SNPs present in NA18562, rs10250 and rs2494735 were also detected here.

FIG. 14. NS results from melanoma 15-plex panel on genomic DNA extracted from fresh frozen melanoma clinical tissue sample. For each amplicon, percentage of variant reads at each nucleotide position corresponding to the human reference genome sequence (hg38) is plotted. The clinical sample had a mutation in BRAF gene, which was detected above the error threshold.

FIGS. 15A-15C. Comparison of the melanoma 15-plex NS panel clinical sample results to Illumina NGS results. FIG. 15A. Summary of sequencing results for 25 clinical melanoma tissue samples (7 fresh/frozen, 18 FFPE). The X-axis shows the VRF based on a standard NGS analysis, and the Y-axis shows the NS VRF using the melanoma BDA panel and LDA. The horizontal line shows the 20% VRF cutoff for NS that was used to make variant calls, and the vertical line shows the 5% VRF cutoff for NGS variant calls. The numbers in quadrants display the number of loci in each group. Importantly, many of the 153 NGS-negative and OCEANS-positive results were true mutations, and as confirmed by ddPCR experiments (FIG. 16C). FIG. 15B. Receiver operator characteristic (ROC) curve for data in FIG. 15A, based on changing the VRF cutoff for NS. The area under the curve (AUC) is 99.99%. FIG. 15C. High concordance of NS results using Oxford Nanopore MinION vs. Flongle flow cells for the 25 melanoma clinical samples.

FIGS. 16A-16C. Confirmation of low VAF mutations with ddPCR. FIG. 16A. NS panel result for BRAF V600K mutation in a FFPE clinical sample. FIG. 16B. ddPCR result for the mutation detected by NS in FIG. 16A. FIG. 16C. Summary of NS and ddPCR comparison experiments for 6 FFPE samples in 4 select mutation loci (BRAF p. V600, KRAS p. G13D, KRAS p. E62K, and MAP2K1 p. P124L). Other than one sample/mutation combination at 31% VAF, ddPCR showed VAFs ranging between 0.02% and 0.66% for the concordant samples.

### DETAILED DESCRIPTION

Provided herein are methods to assemble short DNA molecules (e.g., PCR amplicons) into long, linear concatemers using type IIS restriction enzyme digestion and ligation by DNA ligase. The provided methods and reagents improve assembly length. In nanopore sequencing, the number of DNA molecules that can be sequenced by a flow cell is similar regardless of the length of each DNA molecule, so the provided methods greatly improve the effective throughput of nanopore sequencing. The higher effective sequencing depth can also improve the limit of detection for mutations including single nucleotide variants and small insertions/deletions.

The provided Linear DNA Assembly (LDA) methods use Type IIS restriction enzyme digestion and DNA ligation to assemble many DNA monomers into a long linear concatemer. These methods discourage the formation of circular concatemer products (which cannot be sequenced by NS), (2) do not require assembly of different components in a pre-determined order and are suitable for a variety of NS panels with variable panel sizes, and (3) include several molecular innovations to increase the average length of the assembled concatemer.

I. Linear DNA Assembly

Linear DNA assembly (LDA) using type IIS restriction digestion and ligation requires each DNA monomer molecule to have one end with a type IIS restriction site in the plus (+) orientation (S1) followed by a designed base region (being 2-6 nucleotides in length, e.g., 4 bases) that serves as a sticky end sequence referred to as the Left sticky end sequence (1). The other end of the monomer has the type IIS restriction site in the minus (−) orientation (S1*) followed by a designed base region (being 2-6 nucleotides in length, e.g., 4 bases) that serves as a sticky end sequence referred to as the Right sticky end sequence (1*). The Left and Right sticky end sequences are designed to be complementary to each other.

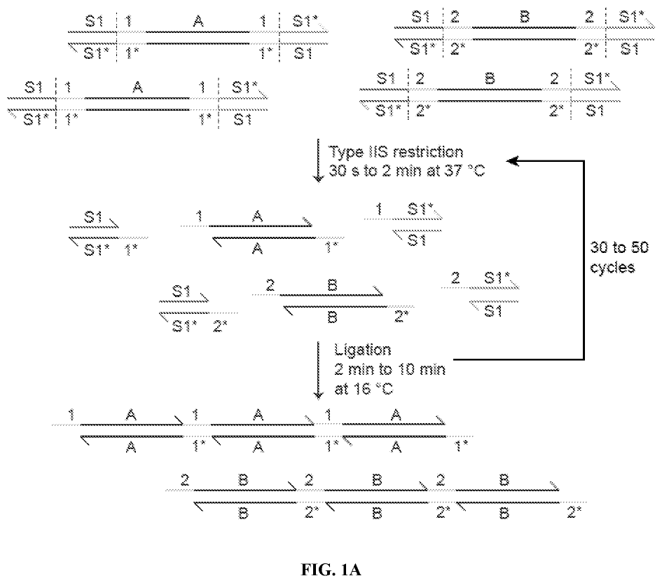

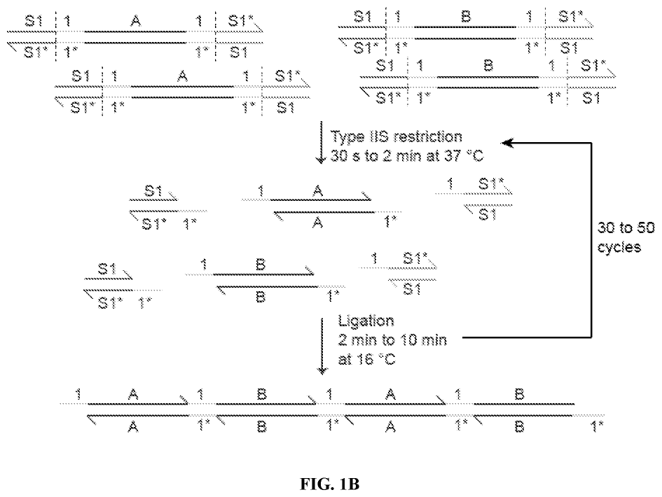

A typical one pot assembly reaction contains 3 pmol to 7 pmol of DNA monomers, 30 U to 60 U of a type IIS restriction enzyme (e.g., BsaI), and 1000 U to 2000 U of DNA ligase (e.g., T4 DNA ligase) in buffer containing 50 mM Tris-HCl, 10 mM MgCl₂, 10 mM DTT, and 1 mM ATP at pH 7.5. As shown in FIGS. 1A-1B, the assembly reaction is carried out by cycling between the optimum temperature for restriction enzyme digestion (37° C.), which is the restriction step, and the optimum temperature for ligation (16° C.), which is the ligation step. At the restriction step, type IIS restriction enzyme cuts at ends of a DNA monomer to generate monomers with sticky ends. During each restriction step, monomers are generated that have either one sticky end (1 or 1*) due to restriction at only one site (S1 or S1*) or two sticky ends (1 and 1*) due to restriction at both sites (S1 and S1*). At the ligation step, complementary sticky ends 1 and 1* hybridize to each other and the DNA ligase enzyme ligates the sticky ends. During each ligation step, two types of hybridization can happen, cross-hybridization between 1 and 1* on different molecules and self-hybridization between 1 and 1* on the same molecule. Cross-hybridization can lead to, two or more monomers ligating together or a monomer ligating to a polymer or two or more polymers ligating together. Cross-hybridization of any two molecules will result in a longer molecule that retains the sticky ends 1 and 1*, allowing the longer molecule to grow further by ligation. Self-hybridization can lead to circularization of a monomer or a polymer molecule. Due to geometrical constraints, the probability of circularization of a monomer should be low, while as the length of a polymer increases its probability of circularization also increases. Self-hybridization of a molecule will terminate its growth and therefore is undesirable for linear DNA assembly. This type of assembly by cycling between restriction and ligation steps is advantageous for assembling long DNA, since in each cycle only a limited number of ligatable monomers are available for a fixed amount of DNA ligase, allowing ligation into long polymers. On the other hand, if a large number of ligatable monomers are available for a fixed amount of DNA ligase, then only dimers and short polymers will be preferred over assembly into long polymers.

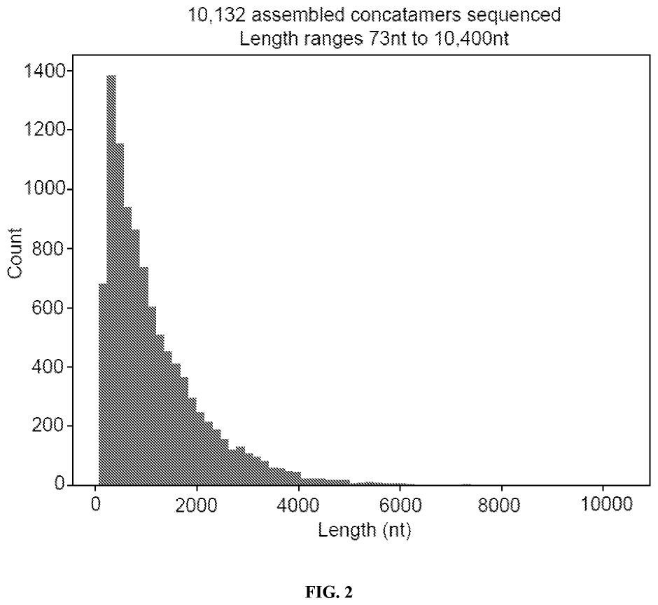

In an exemplary homopolymer assembly reaction (FIG. 1A), DNA monomers of the different families (A or B) have the orthogonal sticky ends (1/1* for family A and 2/2* for family B). This will allow only monomers from the same family to assemble with each other, resulting in homo-polymer assemblies. In an exemplary hetero-polymer assembly reaction (FIG. 1B), DNA monomers of different families have the same sticky ends (1/1* for both family A and B). This will allow monomers from multiple families to assemble with each other, resulting in hetero-polymer assemblies. The data in FIG. 2 show the length distribution of DNA assembled from a 182 bp DNA by LDA. Long, linear fragments of lengths up to 10,400 nt containing 57 monomers can be assembled by this method.

### II. Preparation of DNA Monomers for Assembly

Double-stranded DNA inserts of any size can be assembled by linear DNA assembly, if the required end sequences as mentioned above are present. Adapters containing the end sequences can be added to any DNA insert by dA-tailing and ligation, as shown in FIG. 3A. Ligation by dA-tailing is known to one of ordinary skill in the art of molecular biology. Due to the nature of the adapter ligation, each monomer can only have the same sticky end sequence (1 or 1*) at both ends. So, two different adapters are ligated to the inserts in two separate ligation reactions. In the first ligation reaction, adapter 1, containing restriction site (51) followed by the Left sticky end sequence (1) is ligated to a portion of the insert DNA. In the second ligation reaction, adapter 2, containing restriction site in reverse orientation (S1*) followed by the Right sticky end sequence (1*) is ligated to another portion of the insert DNA. Adapter ligated DNA from both reactions are mixed in equal proportion and used as monomers in the assembly reaction.

End sequences for assembly can be added to any DNA insert by PCR, as shown in FIG. 3B, by using a forward primer that contains the restriction site in (+) orientation (S1), Left sticky end sequence (1), and a DNA insert specific sequence, and a reverse primer that contains the restriction site in (−) orientation (S1*), Right sticky end sequence (1*), and a DNA insert specific sequence. In some embodiments, the primers contain a Unique Molecular Identifier (UMI) barcode sequence between the sticky end sequence (1 or 1*) and the DNA insert specific sequence. The UMI barcode sequences uniquely identify copies of each molecule. When amplicons containing UMIs are sequenced, sequences containing the same UMI can be aligned to correct for PCR errors. The PCR amplicons can be directly used as monomers in the assembly reaction.

### III. Directional Assembly

As mentioned above, self-hybridization of a molecule during assembly can lead to circularization (FIG. 4A). As such, methods to reduce self-hybridization and improve LDA are provided. The methods involve use of a DNA seed that contains a single stranded Left sticky end sequence (1) and a double stranded DNA region devoid of type IIS restriction site (C) (FIG. 4B). Ligatable monomers generated during the restriction step can ligate to the DNA seed by hybridization of the Right sticky end sequence (1*) of the monomer to the Left sticky end sequence (1) of the seed. A polymer formed by such a ligation cannot self-hybridize due to lack of a sticky end sequence at one of its ends. But the polymer can still grow linearly in one direction by cross-hybridization at its Left sticky end sequence (1) to the Right sticky end sequence (1*) of monomers. The polymer formed on a seed cannot hybridize to another seed containing polymer. However, this strategy allows uni-directional assembly of monomers on the DNA seed. To overcome this limitation, the DNA seed design is modified to include two single stranded Left sticky end sequences (1) flanking the double stranded region (C) (FIG. 4C). A polymer formed on such a seed can grow in both directions by cross-hybridization at Left sticky end sequence (1) at both ends to the Right sticky end sequence (1*) of monomers. To get long assemblies, the seed is mixed at low relative concentrations of 0.05X to 0.01X of the monomer concentration in the assembly reaction. If the seed concentration is high, then ligation of individual seeds to a monomer will exhaust the Right sticky end sequence (1*) of the monomers, leaving only short polymers with only the Left sticky end sequence (1), thereby, inhibiting cross-hybridization and linear growth of the polymers. At low relative concentrations of seed, ligation of individual seeds to a monomer will still leave sufficient Right sticky end sequence (1*) of monomers available for assembly on to the seed containing polymers to form longer assemblies. Since, longer assemblies have a higher probability for circularization, assembly on DNA seeds will increase the fraction of long linear assemblies. Though seeds are present at low initial concentrations, after assembly a significant fraction of the assemblies will be formed on the seed. For example, with a starting seed concentration of 0.05X (5%), if it is assumed that on average 8 monomers are assembled into a polymer, then after assembly 8*5%=40% of assemblies will be on a seed.

IV. Blocking Side Product Assembly

During the restriction step of the assembly reaction, in addition to monomers with sticky ends (1 and 1*) two side products, SP1 and SP2 with Right sticky end sequence (1*) and Left sticky end sequence (1) respectively are formed (FIG. 5A). These side products can be ligated to a growing assembly during the ligation step. Once this occurs, assembly on the end, where the side product ligated is stopped. But, ligation of the side product will lead to regeneration of the functional restriction site on the assembly. So, restriction by the type IIS enzyme is necessary to allow the growth of the assembly from that end. These side products accumulate over the course of the reaction and can significantly inhibit the linear assembly of monomers. To overcome this limitation, single stranded destructive probes that are complementary to the strand on the side product that contains the sticky end sequence (1 or 1*) can be used (FIG. 5B). The sticky end sequence (1) on destructive probe 1 acts as a toehold and reacts with the side product SP1, while destructive probe 2 reacts with SP2 using its (1*) sticky end sequence to displace one of the original strands on the side products forming a fully double-stranded product (P1 and P2). P1 and P2 lack any sticky end sequence but still have a functional restriction site that the type IIS enzyme can recognize and cut on the destructive strand to generate SP1 and SP2 again. To prevent this, the destructive probes can have modifications at the cut site that will inhibit cutting by the type IIS enzyme. Modifications include, but are not limited to, DNA with phosphorothioate-substituted backbone, sugar modified nucleotides like 2′Fluoro and 2′-0Me, inverted DNA nucleotides, methylated bases, and DNA with carbon or polyethylene glycol (PEG) spacers can be used. Destructive probes are added at high relative concentrations of 10X to 50X of the side product, to favor the formation of P1 and P2 over the assembly of the side products into a growing polymer or monomer.

Preliminary NS analysis of read length was performed for a 182 bp DNA assembled by LDA on a DNA seed by bi-directional assembly and using destructive probes (Table 1). The use of a DNA seed and destructive probes improved the length of the assembled DNA by around 56% compared to normal LDA.

Table 1. NS data showing increase in read length by LDA with seed and destructive probes

| | | | LDA with seed |
|---|---|---|---|
| | | LDA with | and destructive |
| | LDA | seed | probes |
| No. of reads with seed | — | 551 | 420 |
| No. of reads without | 1969 | 1499 | 1425 |
| seed | | | |
| Avg. length with seed | — | 1170 | 1238 |
| Avg. length without | 801 | 685 | 727 |
| seed | | | |

### V. Operation of LDA for Long-read Sequencing

Long-read sequencing platform based on nanopores like Oxford Nanopore Sequencing (NS) have several advantages over short-read sequencers (e.g., Illumina) such as being able to produce real-time data, rapid library prep, portability, and low capital cost. But NS suffers from a higher intrinsic error rate of roughly 10% compared to 0.2% for Illumina and also produces lower number of reads compared to Illumina. This prevents the use of NS for rare variant detection. Variant enrichment strategies that can enrich rare variants over the intrinsic error rate of NS can potentially enable use of NS for rare variant detection. Variant enrichments methods like Blocker Displacement Amplification (BDA), ICE COLD PCR, or PNA-blocker PCR produces short amplicons of 100 bp - 300 bp in length. PCR that produces short amplicons are routinely used in a number of diagnostic assays like cell-free DNA (cfDNA) analysis and in assays designed for short-read sequencing platforms. But sequencing short DNA (<300 bp) on NS produces reads of low quality and yield. Linear DNA assembly to assemble short DNA into long assemblies can enable NS to produce reads of higher quality and yield for short amplicon sequencing.

NS can sequence ultra-long reads up to several Mbs in size. Therefore, higher order assemblies of short amplicons are needed to utilize the full potential of NS. In molecular cloning, assembly by type IIS restriction and ligation (i.e., Golden Gate assembly) is one method for cloning up to 20 inserts into vectors. Gibson assembly, which is another method for cloning is used for cloning up to only 5 inserts, due to lower efficiency of assembly for higher number of inserts. Gibson assembly also requires longer sticky ends around 20 bases in length. This requires two separate PCR reactions to attach end sequences for assembly on to DNA inserts. Since, forward and reverse primers with 20-base complementarity will form primer dimers even at elevated temperatures of around 55° C.-72° C. that are typically used during the annealing and extension step of PCR and impair PCR amplification of the desired DNA insert. In the LDA method provided herein, sticky ends (1 and 1*) are only 4 bases long and hence will not form primer dimers during the annealing and extension steps of PCR. As such, LDA-adapter forward and reverse primers designed as depicted in FIG. 3B can directly be used in a single PCR to generate amplicons for assembly. In applications like BDA, where the primers cannot be modified to have the type IIS restriction site and sticky-end sequences at 5′end, the amplicons generated from BDA can be used as input in a single second PCR to attach end sequences.

Circularization of short DNA and Rolling Circle Amplification (RCA) of the circular DNA can generate long single stranded DNA (ssDNA) composed of multiple copies (up to 50 copies) of the same DNA sequence. But NS cannot sequence ssDNA directly, since dsDNA sequencing adaptors containing bound motor proteins are ligated to ends of DNA to be sequenced. The motor proteins are needed for translocation of DNA through the nanopore for sequencing. Even if the ends of the DNA are made double stranded by hybridizing short oligos to the ends, the presence of significant structure in the ssDNA region of the RCA product interferes with NS. To generate dsDNA from RCA, random hexamers can be used during RCA. But this method generates highly branched DNA that needs to be debranched before NS. The presence of random hexamers also generates non-specific amplification products. Therefore, though RCA can generate long DNA fragments from short DNA, it is laborious involving multiple steps (circularization, exonuclease digestion to remove linear DNA, RCA and conversion to dsDNA/debranching) making it incompatible for rapid library preparation for NS. Therefore, the LDA method provided herein, which can rapidly assemble short amplicons into relatively long assemblies, is ideally suited for NS.

### VI. Shortening NS Library Preparation Time by LDA with Barcode Adapter Seeds

Library preparation for NS involves ligating barcodes for sample identification followed by ligation of an NS adapter containing a motor protein. The motor protein on the NS adapter is necessary to regulate the speed of DNA translocation through the nanopore for proper interpretation of the DNA sequence. As depicted in FIG. 8A, this is normally done in three steps:
1. End prep of DNA, which involves phosphorylating 5′ ends of DNA and adding dA overhang to the 3′ ends of DNA;
2. Ligation of barcoded adapter containing a unique barcode (BC) and a sticky end sequence (2) for NS adapter ligation; and
3. Ligation of NS adapter.

Here, the provided methods shorten NS library preparation time. The methods involve use of a barcode adapter seed that contains a single stranded Left sticky end sequence (1), a double stranded DNA region devoid of type IIS restriction site but containing a unique barcode sequence for sample identification (BC), and a Right sticky end sequence (2) for NS adapter ligation (FIG. 8B). During LDA, ligatable monomers generated during the restriction step can ligate to the seed by hybridization of the Right sticky end sequence (1*) of the monomer to the Left sticky end sequence (1) of the seed. A polymer formed by such a ligation cannot self-hybridize due to lack of a compatible sticky end sequence at one of its ends. But the polymer can still grow linearly in one direction by cross-hybridization at its Left sticky end sequence (1) to the Right sticky end sequence (1*) of monomers. The DNA assembled by this method will have the barcode sequence and also the sticky end sequence (2) at one end for NS adapter ligation. Therefore, DNA from LDA can directly be used to ligate NS adapter for sequencing. This method eliminates the end prep (step 1) and barcode ligation (step 2) from the library preparation workflow, shortening the library preparation time by 2-3 hours. Table 2 shows preliminary NS run data from a library prepared using barcode adapter seed in LDA. Throughput, Q-score, and length of LDA assembled DNA are compared to normal LDA and library preparation. There was a —15% decrease in reads with barcode compared to normal library preparation.

Table 2. Comparison of NS by normal library prep after LDA and shortened library prep after LDA with barcode adapter seed

| | | LDA with Barcode |
|---|---|---|
| | LDA | adapter seed |
| No. of reads in 1 hour | 258,000 | 307,290 |
| Average Q-score | 10.1 | 9.98 |
| Average length | 1135 | 1017 |
| % Reads with | 96% | 82% |
| Barcode | | |

### VII. Application Considerations for Low VAF Detection Using NS

Low VAF detection is essential for diagnostic applications in cancer.

Commercial tests based on the Illumina platform, such as FoundationOne and whole exome sequencing, for analysis of tumor mutation burden provide detailed information on potential pathogenic mutations for guiding therapy selection. However, short-read sequencers like Illumina are less suitable for the analysis of large deletions, fusions, and copy number variations. In addition, library preparation for Illumina sequencing typically takes 24 hours, with the sequencing run taking another 2 days and bioinformatic interpretation taking 1-2 days. Consequently, analysis of cancer samples can take a minimum of 4 days from sample to answer. Illumina instruments also require significant capital investment. As such, samples have to be sent to a centralized location for sequencing, which adds additional time for sample processing. These limitations can be overcome by enabling low VAF detection on the NS platform. NS is already well-suited for the analysis of DNA structural variants and copy number variants due to its long-read capability. Adding the capability of low VAF detection to NS will make it the preferred platform for rapid and comprehensive analysis of cancer genomics.

The variant enrichment method, BDA (Wu et al., 2017; US 2017/0067090; and WO 2019/164885, each of which is incorporated herein by reference in its entirety), was combined with LDA (FIG. 9) to enable detection of VAF as low as 0.1% on NS. This is a 200-fold improvement on the current VAF detection of NS. In short, a typical BDA system uses 3 different oligos: a forward primer, a reverse primer, and a blocker. The forward and reverse primers function as standard PCR primers. The forward primer and blocker are designed to have a certain degree of sequence overlap (e.g., between 4 and 15 nucleotides), such that binding of the forward primer and blocker to the template DNA will be mutually exclusive. The system is designed such that the blocker binds to a wild-type DNA template with perfect match and to the variant DNA with mismatch. As such, displacement of the blocker by the forward primer binding to a variant DNA template is energetically favorable under standard PCR conditions (e.g., the standard free energy of the forward primer displacing the blocker at 60° C. in 5 mM Mg′ is between 0 kcal/mol and +5 kcal/mol). This leads to preferential amplification of the variant DNA over wild-type DNA in each PCR cycle. Amplicons from a typical BDA reaction can be used as the template for PCR with LDA-adapter primers as shown in FIG. 9 to generate monomers for LDA. Amplicons can then be assembled by LDA and used in NS library preparation and sequencing.

### VIII. Application Considerations for Mutation Detection in Cancer

Low VAF detection on NS demonstrated above and in Example 3 makes NS suitable for mutation detection in cancer. Acute Myeloid Leukemia (AML) is a type of blood cancer in which the bone marrow produces abnormal red blood cells, platelets or myelobalsts. Previously, NS could detect only mutations with >20% VAF because of its high error rate. A 7-plex NS AML panel was designed for detecting mutations in 6 genes at 7 loci, which are involved in AML with a sensitivity of 1% VAF. Mutations in all 7 loci are detected in a single multiplex-reaction following the workflow in FIG. 9 except that 7 different amplicons were present in the BDA reaction and 7 different LDA-adapter primer pairs containing different gene specific regions but the same LDA site (S1,1 & S1,1*) were used in the PCR to attach LDA sites to amplicons. The amplicons are then assembled by LDA as heteropolymers. The panel was able to detect mutations in FLT3, DNMT3A, IDH1, KIT, NPM1, and IDH2 (2 loci), genes that are known to be involved in AML. Detecting these mutations can help in diagnosis and designing treatment plans for patients with AML. FIG. 11 shows results with synthetic genes carrying AML mutations spiked in at 1% VAF in human genomic DNA (NA18537) background. Mutations at all 7 loci in the 6 genes were detected at 1% VAF. The panel was further validated on a human cancer cell-line (HD892) genomic DNA sample carrying mutations in NPM1, DNMT3A, IDH1, FLT3, and one locus of IDH2 at 5% VAF (FIG. 12). All 5 mutations were detected by the panel, while no mutations were detected in KIT and the second locus of IDH2 where the cell-line sample does not contain mutations.

Melanoma is a type of skin cancer in which pigment producing cells called melanocytes become mutated causing cancer. A 15-plex NS melanoma panel was designed for detecting mutations in 9 genes at 15 loci, which are involved in melanoma with sensitivity of 1% VAF in a single reaction. The panel can detect mutations in MAP2K1, MAP2K2, AKT1, AKT3, NRAS, KRAS, PIK3CA, and BRAF genes. FIG. 13 shows results with synthetic genes carrying melanoma mutations spiked in at 1% VAF in human genomic DNA (NA18562) background. Mutations at all 15 loci in the 9 genes were detected at 1% VAF.

The panel was further tested on genomic DNA extracted from a fresh frozen melanoma clinical tissue sample (FIG. 14). BRAF V600E mutation, which is common in melanoma patients, was detected. The presence of this mutation was also verified by NGS.

Next, the melanoma panel was applied to 25 clinical melanoma tissue samples, including both fresh/frozen (FF) and FFPE tissue (FIG. 15A). Somatic mutations were called only when the VRF was observed to be greater than 20%. In total, DNA from 7FF and 18 FFPE tissue samples were sequenced using both NS and NGS. The melanoma NS panel covers a total of 384 loci, corresponding to a total of 9600 total loci analyzed across the 25 samples. FIG. 15A shows the comparison between NS and NGS. All 16 somatic mutants called by NGS at above 5% VAF were also called by NS, corresponding to a 100% NS sensitivity relative to NGS. Of the 9584 NGS-negative loci, OCEANs called an additional 153 variants (FIG. 15A); thus, relative to NGS, the NS panel had a 99.0% specificity. By varying the VRF cutoff threshold, the number of variant calls by NS can be changed, generating a set of sensitivity/specificity tradeoffs, which can be plotted as a receiver-operator characteristic (ROC) curve (FIG. 15B). The area under the ROC curve is 99.99%, indicating very high concordance between the NS panel and NGS when the NS variant LoD is artificially weakened by setting higher VRF thresholds.

Importantly, many of the 153 discordant called variants based on a 20% VRF threshold could be real mutations missed by NGS. To confirm the discordant NS mutation calls, droplet digital PCR (ddPCR) was performed on 6 FFPE samples at 4 mutation loci (BRAF p. V600, KRAS p. G13, KRAS p. E62, and MAP2K1 p. P124). Of these 24, 11 mutations were called positive by NS, and 13 were called negative by NS. NS was concordant with ddPCR for 10 positive samples and 11 negative samples (FIG. 16C).

Next, the reproducibility and robustness of the NS panel was characterized on different types of nanopore sequencing instruments and flow cells. The Oxford Nanopore Flongle flow cell, in particular, is relatively inexpensive at $90, and can further reduce turnaround time relative to MinION by reducing the need for sample batching before sequencing. The NS panel was performed on all 25 melanoma samples on the Flongle. Highly quantitatively similar VRFs were observed as compared to the MinION (FIG. 15C).

These results show that BDA combined with LDA can enable NS to be used for cancer mutation profiling in the clinic.

### IX. Interpretation of NS Data that Utilize BDA and LDA

An embodiment of an algorithm to analyze NS reads from FASTS files is described below. Similar algorithms from FASTS or FASTQ files can similarly be constructed by one of ordinary skill in the art of bioinformatic processing of sequencing data.
1. Remove reads with low quality score (e.g., Q score <7)
2. Convert FASTS to FASTQ file
3. For each read, look for the junction site of assembly and deconcatenate reads at the assembly site to generate individual sequences
4. Align individual sequences to reference amplicon sequence to generate a sam/bam file
5. Analyze the number of reads mapped to the wild type sequence at each position and calculate the variant percent at each position
6. Call variant at a position if the variant percentage at that position is above a certain threshold that depends on the error rate of NS for that amplicon sequence (e.g., >40%)

### X. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1— LDA Improves NS Read Throughput and Quality

The results presented in FIG. 6A, show NS read throughput comparison for DNA monomer library sequenced without LDA (mean length 317 nt) and with LDA (mean length 1577 nt). The read throughput with LDA (250,000 per hour) is only slightly less than without LDA (300,000 per hour). But, with LDA each read contains on average 5 monomers, so the actual throughput is 1.25 million per hour, which is over 4-fold higher than without LDA. The NS read quality-score comparison in FIG. 6B shows that LDA also significantly improves the quality of reads.

### Example 2— Variant Allele Detection by NS after LDA

FIGS. 7A-7B show the NS sequencing results for PCR amplicons designed to cover two SNPs, rs3789806(C>G) and rs9648696(T>C) in human genomic DNA (gDNA). When the fraction of reads at each nucleotide position mapping to the wild type allele is plotted (top panels of FIG. 7A and FIG. 7B), the SNPs could not be identified in the 5% variant sample due to the high intrinsic error rate of NS. Background subtraction of the matched 0% variant sample from the 5% variant sample could be used to correct for the NS error rate. Therefore, the fraction of reads mapped to the highest frequency variant allele in the 5% variant sample, minus the variant allele frequency in the matched normal 0% variant sample (ΔVariant allele %) is plotted (bottom panels of FIG. 7A and FIG. 7B). Even after background subtraction, direct NS of amplicon without LDA could not detect the SNPs due to false positive signals at other positions (bottom panel of FIG. 7A). LDA assembled reads enable confident detection of the two SNPs by this method (bottom panel of FIG. 7B). The higher read depth and quality of NS reads after LDA enabled detection of the two SNPs at 5% VAF in human gDNA samples after background subtraction, thereby demonstrating the utility of LDA for variant allele detection by NS.

### Example 3— Detection of 0.1% VAF by NS after LDA using BDA

Amplicons from a typical BDA reaction were used as the template for PCR with LDA-adapter primers as shown in FIG. 9 and Table 3 to generate monomers for LDA. Amplicons assembled by LDA were used in NS library preparation and sequenced on the Minion device from Oxford Nanopore Technologies for 1 hour. FIG. 10 shows preliminary results in which two SNPs rs3789806(C>G) and rs9648696(T>C) at 0.1% VAF are detected in human gDNA samples of 0% and 0.1% NA18562 in NA18537 using NS. When fraction of reads at each nucleotide position mapping to the wild-type allele is plotted (top panel of FIG. 10), the SNPs could be identified in the 0.1% variant sample. Therefore, combining BDA with LDA enabled low VAF detection on NS without the requirement for background subtraction. Thus, a matched 0% variant sample is not necessary for low VAF detection using NS by this method.

### Example 4— AML 7-Plex Panel

ow VAF detection on NS demonstrated above and in Example 3 makes NS suitable for mutation detection in cancer. Acute Myeloid Leukemia (AML) is a type of blood cancer in which the bone marrow produces abnormal red blood cells, platelets or myelobalsts. Previously, NS could detect only mutations with >20% VAF because of its high error rate. A 7-plex NS AML panel was designed for detecting mutations in 6 genes at 7 loci, which are involved in AML with a sensitivity of 1% VAF. Mutations in all 7 loci are detected in a single multiplex-reaction following the workflow in FIG. 9 and the primers in Table 3, except that 7 different amplicons were present in the BDA reaction and 7 different LDA-adapter primer pairs containing different gene specific regions but the same LDA site (S1,1 & S1,1*) were used in the PCR to attach LDA sites to amplicons. The amplicons are then assembled by LDA as heteropolymers. The panel was able to detect mutations in FLT3, DNMT3A, IDH1, KIT, NPM1, and IDH2 (2 loci), genes that are known to be involved in AML. Detecting these mutations can help in diagnosis and designing treatment plans for patients with AML. FIG. 11 shows results with synthetic genes carrying AML mutations spiked in at 1% VAF in human genomic DNA (NA18537) background. Mutations at all 7 loci in the 6 genes were detected at 1% VAF. The panel was further validated on a human cancer cell-line (HD892) genomic DNA sample carrying mutations in NPM1, DNMT3A, IDH1, FLT3, and one locus of IDH2 at 5% VAF (FIG. 12). All 5 mutations were detected by the panel, while no mutations were detected in KIT and the second locus of IDH2 where the cell-line sample does not contain mutations.

### Example 5— Melanoma 15-Plex Panel

Melanoma is a type of skin cancer in which pigment producing cells called melanocytes become mutated causing cancer. A 15-plex NS melanoma panel (Table 3) was designed for detecting mutations in 9 genes at 15 loci, which are involved in melanoma with sensitivity of 1% VAF in a single reaction. The panel can detect mutations in MAP2K1, MAP2K2, AKT1, AKT3, NRAS, KRAS, PIK3CA, and BRAF genes. FIG. 13 shows results with synthetic genes carrying melanoma mutations spiked in at 1% VAF in human genomic DNA (NA18562) background. Mutations at all 15 loci in the 9 genes were detected at 1% VAF. The panel was further tested on genomic DNA extracted from a fresh frozen melanoma clinical tissue sample (FIG. 14). BRAF V600E mutation, which is common in melanoma patients, was detected. The presence of this mutation was also verified by NGS.

Next, the melanoma panel was applied to 25 clinical melanoma tissue samples, including both fresh/frozen (FF) and FFPE tissue (FIG. 15A). Somatic mutations were called only when the VRF was observed to be greater than 20%. In total, DNA from 7 FF and 18 FFPE tissue samples were sequenced using both NS and NGS. The melanoma NS panel covers a total of 384 loci, corresponding to a total of 9600 total loci analyzed across the 25 samples. FIG. 15A shows the comparison between NS and NGS. All 16 somatic mutants called by NGS at above 5% VAF were also called by NS, corresponding to a 100% NS sensitivity relative to NGS. Of the 9584 NGS-negative loci, OCEANs called an additional 153 variants (FIG. 15A); thus, relative to NGS, the NS panel had a 99.0% specificity. By varying the VRF cutoff threshold, the number of variant calls by NS can be changed, generating a set of sensitivity/specificity tradeoffs, which can be plotted as a receiver-operator characteristic (ROC) curve (FIG. 15B). The area under the ROC curve is 99.99%, indicating very high concordance between the NS panel and NGS when the NS variant LoD is artificially weakened by setting higher VRF thresholds.

Importantly, many of the 153 discordant called variants based on a 20% VRF threshold could be real mutations missed by NGS. To confirm the discordant NS mutation calls, droplet digital PCR (ddPCR) was performed on 6 FFPE samples at 4 mutation loci (BRAF p. V600, KRAS p. G13, KRAS p. E62, and MAP2K1 p. P124). Of these 24, 11 mutations were called positive by NS, and 13 were called negative by NS. NS was concordant with ddPCR for 10 positive samples and 11 negative samples (FIG. 16C).

Next, the reproducibility and robustness of the NS panel was characterized on different types of nanopore sequencing instruments and flow cells. The NS panel was performed on all 25 melanoma samples on the Oxford Nanopore Flongle flow cell. Highly quantitatively similar VRFs were observed as compared to the MinION (FIG. 15C).

These results show that BDA combined with LDA can enable NS to be used for cancer mutation profiling in the clinic.

TABLE 3
Oligos used for NS using combined BDA and LDA

| | | | SEQ |
|---|---|---|---|
| | | | ID |
| Gene | Design | Sequence | NO |
| rs3789806 | FP | CTTGTATATAGACGGTAAAATAAACACCAA | 1 |
| | | GA | |
| | RP | AGGCACCAGAAGTCATCAGAATG | 2 |
| | B | TAAACACCAAGACGTGGTAAATATTTACCT | 3 |
| | | GGT/iSpC3//iSpC3/CG | |
| FLT3 | FP | CCCTGACAACATAGTTGGAATCA | 4 |
| | RP | ACTCCAGGATAATACACATCACAGT | 5 |
| | B | TGGAATCACTCATGATATCTCGAGCCA/SpC | 6 |
| | | 3//iSpC3/CG | |
| DNMT3A | FP | AGCAGTCTCTGCCTCGC | 7 |
| | RP | AGAAGATTCGGCAGAACTAAGCA | 8 |
| | B | CCTCGCCAAGCGGCTCATGTT/iSpC3//iSpC3/ | 9 |
| | | AC | |
| IDH1 | FP | GCTTGTGAGTGGATGGGTAAAAC | 10 |
| | RP | TGTGTTGAGATGGACGCCTATT | 11 |
| | B | TGGGTAAAACCTATCATCATAGGTCGTCAT | 12 |
| | | G/iSpC3//iSpC3/TC | |
| IDH2_140 | FP | AGAAGATGTGGAAAAGTCCCAATG | 13 |
| | RP | GTGCCCAGGTCAGTGGAT | 14 |
| | B | TCCCAATGGAACTATCCGGAACATCC/iSpC3 | 15 |
| | | //iSpC3/GC | |
| IDH2_172 | FP | CTGGTCGCCATGGGCGT | 16 |
| | RP | TGAAGAAGATGTGGAAAAGTCCCA | 17 |
| | B | GGCGTGCCTGCCAATGGTGA/iSpC3//iSpC3/G | 18 |
| | | A | |
| KIT | FP | TCCTTTAACCACATAATTAGAATCATTCTTG | 19 |
| | | A | |
| | RP | AGTTAGTTTTCACTCTTTACAAGTTAAAAT | 20 |
| | | GA | |
| | B | ATCATTCTTGATGTCTCTGGCTAGACCAAA/ | 21 |
| | | iSpC3//iSpC3/CT | |
| NPM1 | FP | GTTTAAACTATTTTCTTAAAGAGACTTCCTC | 22 |
| | | C | |
| | RP | TTAAAGTGTTTGGAATTAAATTACATCTGA | 23 |
| | | GT | |
| | B | ACTTCCTCCACTGCCAGAGATCTTGAA/SpC | 24 |
| | | 3//iSpC3/GG | |
| MAP2K1_57 | FP | TTGAGGCCTTTCTTACCCA | 25 |
| | RP | GGCTTGTGGGAGACCTTGA | 26 |
| | B | TCTTACCCAGAAGCAGAAGGTGGGA/iSpC3// | 27 |
| | | iSpC3/AA | |
| MAP2K1_121 | FP | AGCTGCAGGTTCTGCAT | 28 |
| | RP | AGCCACCCAACTCTTAAGGC | 29 |
| | B | CTGCATGAGTGCAACTCTCCGTACA/iSpC3//i | 30 |
| | | SpC3/GC | |
| MAP2K1_203 | FP | CGCTGACCCCAAAGTCACA | 31 |
| | RP | AGTTCCCTCCTTTTCTATTTTCTCTTC | 32 |
| | B | AAAGTCACAGAGCTTGATCTCCCCAC/iSpC3 | 33 |
| | | //iSpC3/GC | |
| MAP2K2_57 | FP | TTCGCCGACCTTGGCT | 34 |
| | RP | AGTCTCCCTAGGTAGCTAACCC | 35 |
| | B | TTGGCTTTCTGGGTGAGAAAGGCTT/iSpC3//i | 36 |
| | | SpC3/AC | |
| MAP2K2_125 | FP | CTGCAGGTCCTGCACGA | 37 |
| | RP | GGACGCACTCACCATGTGT | 38 |
| | B | CTGCACGAATGCAACTCGCCGTA/iSpC3//iSp | 39 |
| | | C3/TA | |
| MAP2K2_207 | FP | CATCCTCGTGAACTCTAGAGG | 40 |
| | RP | GGGACTCACAGCCATGTAGG | 41 |
| | B | TCTAGAGGGGAGATCAAGCTGTGTGA/iSpC | 42 |
| | | 3//iSpC3/AA | |
| AKT1 | FP | AACACCTTCATCATCCGCT | 43 |
| | RP | CCATCCCCGTGTCCCTC | 44 |
| | B | CGCTGCCTGCAGTGGACCACT/iSpC3//iSpC3/ | 45 |
| | | CC | |
| AKT3 | FP | TCAAAAGGAAGTATCTTGGCCTCC | 46 |
| | RP | CCAGTGTTGTAGGACATATATTGTACC | 47 |
| | B | GCCTCCAGTTTTTTATATATTCTCCTACATG | 48 |
| | | AGG/iSpC3//iSpC3/AA | |
| NRAS_12 | FP | CAGTGCGCTTTTCCCAACA | 49 |
| | RP | GCTTTAAAGTACTGTAGATGTGGCTC | 50 |
| | B | CCCAACACCACCTGCTCCAACC/iSpC3//iSpC | 51 |
| | | 3/CT | |
| NRAS_61 | FP | TTGTTGGACATACTGGATACAGC | 52 |
| | RP | GGTTAATATCCGCAAATGACTTGC | 53 |
| | B | GATACAGCTGGACAAGAAGAGTACAGTG/iS | 54 |
| | | pC3//iSpC3/AC | |
| KRAS_12 | FP | GTCAAGGCACTCTTGCCTAC | 55 |
| | RP | TGTATTAACCTTATGTGTGACATGTTCTAA | 56 |
| | B | TGCCTACGCCACCAGCTCCA/iSpC3//iSpC3/T | 57 |
| | | T | |
| KRAS_61 | FP | TCTTGGATATTCTCGACACAGCA | 58 |
| | RP | TTATATTCAATTTAAACCCACCTATAATGG | 59 |
| | | TG | |
| | B | CACAGCAGGTCAAGAGGAGTACAGTG/iSpC | 60 |
| | | 3//iSpC3/AC | |
| PIK3CA_542 | FP | CAATTTCTACACGAGATCCTCTCT | 61 |
| | RP | GGTATGGTAAAAACATGCTGAGATCA | 62 |
| | B | ATCCTCTCTCTGAAATCACTGAGCAGG/iSpC | 63 |
| | | 3//iSpC3/AC | |
| PIK3CA_1047 | FP | TTGGAGTATTTCATGAAACAAATGAATGAT | 64 |
| | RP | CAGTGCAGTGTGGAATCCAG | 65 |
| | B | CAAATGAATGATGCACATCATGGTGGC/iSp | 66 |
| | | C3//iSpC3/GT | |
| BRAF_600 | FP | GGACCCACTCCATCGAGAT | 67 |
| | RP | TTACTTACTACACCTCAGATATATTTCTTCA | 68 |
| | | TG | |
| | B | CCATCGAGATTTCACTGTAGCTAGACCAAA | 69 |
| | | A/iSpC3/iSpC3/AA | |
| LDA-adapter | FP | CAATTCGGTCTCCAGTG-Gene specific | 70 |
| | | sequence | |
| | RP | CAATTCGGTCTCCCACT-Gene specific | 71 |
| | | sequence | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
US 2017/0067090
WO 2019/164885
Wu et al., (2017). Multiplexed enrichment of rare DNA variants via sequence-selective and temperature-robust amplification. Nature Biomedical Engineering, 1(9), 714-723.

What is claimed is:

1. An aqueous solution for DNA monomer assembly, the solution comprising:
a plurality of double-stranded DNA monomer species, each monomer species comprising, from 5′ to 3′:
a type IIS restriction site in the (+) orientation (S1),
a designed Left sticky end DNA sequence (1),
an insert sequence (A),
a second designed Right sticky end DNA sequence (1*), and
a type IIS restriction site in the (−) orientation (S1*),
wherein at least two different DNA monomers comprise the same Left sticky end DNA sequence, wherein at least two different DNA monomers comprise the same Right sticky end DNA sequence, and wherein the Left sticky end DNA sequence and the Right sticky end DNA sequence are complementary to and can form Watson-Crick base pairs with each other;
a type IIS DNA restriction enzyme;
a DNA ligase enzyme; and
a chemical buffer suitable for the enzymatic functions of the type IIS DNA restriction enzyme and the DNA ligase enzyme.

2. The solution of claim 1, further comprising a partially double-stranded DNA seed molecule, the seed molecule comprising, from 5′ to 3′:
a single-stranded Left sticky end DNA sequence (1); and
a double stranded DNA region devoid of a type IIS restriction site (C).

3. The solution of claim 1, further comprising a partially double-stranded DNA seed molecule, the seed molecules comprising, from 5′ to 3′:
a Left sticky end DNA sequence (1);
a double stranded DNA region devoid of a type IIS restriction site (C); and
a Left sticky end DNA sequence (1).

4. The solution of any one of claims 1-3, wherein the chemical buffer comprises
between 20 mM and 150 mM Tris-HCl,
between 2 mM and 50 mM MgCl2,
between 0 mM and 50 mM DTT, and
between 0.1 mM and 10 mM ATP,
wherein the buffer exhibits a pH between 5.5 and 9.5 at 25° C.

5. The solution of any one of claims 1-3, wherein the type IIS DNA restriction enzyme is selected from BsaI, BbsI, BsmBI, BtgZI, Esp3I, and SapI, wherein the S1 and S1* restriction sites correspond to the recognition site of the type IIS DNA restriction enzyme selected, and wherein the concentration of the type IIS DNA restriction enzyme is between 0.15 U/μL and 15 U/μL.

6. The solution of any one of claims 1-3, wherein the DNA ligase enzyme is selected from T4 DNA ligase, T7 DNA ligase, T3 DNA ligase, Taq DNA ligase, and E. coli DNA ligase, and wherein the concentration of the DNA ligase is between 5 U/μL and 500 U/μL.

7. The solution of any one of claims 1-3, wherein the Left sticky end DNA sequence and the Right sticky end DNA sequence each have a length of 2-6 nucleotides.

8. The solution of any one of claims 1-3, wherein the insert sequence of each monomer has a length between 40 nt and 2,000 nt.

9. The solution of any one of claims 1-3, wherein the total concentration of all DNA monomers is between 5 nM and 5 μM.

10. The solution of claim 2 or 3, wherein the total concentration of all DNA monomers is 1x to 1000x the concentration of partially double-stranded DNA seed molecules.

11. A method for linear assembly of DNA concatemers from a plurality of double-stranded DNA monomers, each monomer species comprising, from 5′ to 3′:
a type IIS restriction site in the (+) orientation (S1),
a designed Left sticky end DNA sequence (1),
an insert sequence (A),
a second designed Right sticky end DNA sequence (1*), and
a type IIS restriction site in the (−) orientation (S1*);
wherein at least two different DNA monomers comprise the same Left sticky end DNA sequence, wherein at least two different DNA monomers comprise the same Right sticky end DNA sequence, and wherein the Left sticky end DNA sequence and the Right sticky end DNA sequence are complementary to and can form Watson-Crick base pairs with each other;
the method comprising:
mixing the DNA monomers with a type IIS DNA restriction enzyme, a DNA ligase enzyme, and a chemical buffer suitable for the enzymatic functions of the type IIS DNA restriction enzyme and the DNA ligase enzyme; and
thermal cycling the solution between 5 cycles and 100 cycles, with each cycle comprising between 5 seconds and 5 minutes at a temperature between 30° C. and 45° C., and between 30 seconds and 30 minutes at a temperature between 10° C. and 25° C.

12. The method of claim 11, wherein a partially double-stranded DNA seed molecule is mixed with the monomer molecules before thermal cycling, the seed molecule comprising, from 5′ to 3′:
a single-stranded Left sticky end DNA sequence (1); and
a double stranded DNA region devoid of a type IIS restriction site (C).

13. The method of claim 11, wherein a partially double-stranded DNA seed molecule is mixed with the monomer molecules before thermal cycling, the seed molecule comprising, from 5′ to 3′:
a Left sticky end DNA sequence (1);
a double stranded DNA region devoid of a type IIS restriction site (C); and
a Left sticky end DNA sequence (1).

14. The method of claim 11, wherein a partially double-stranded DNA seed molecule is mixed with the monomer molecules before thermal cycling, the seed molecule comprising, from 5′ to 3′:
a Left sticky end DNA sequence (1);
a double stranded DNA region devoid of a type IIS restriction site (C) and a unique barcode; and
a sticky end DNA sequence (2) for appending adapters for nanopore sequencing.

15. The method of claim 11, wherein the DNA monomers are generated by a method comprising:
amplifying a DNA template by multiplex polymerase chain reaction (PCR) amplification, comprising:
adding to a DNA template solution:
a set of forward DNA primers comprising, from 5′ to 3′:
a type IIS restriction site in the (+) orientation (S1),
a designed Left sticky end DNA sequence (1), and
a gene-specific sequence;
a set of reverse DNA primers comprising, from 5′ to 3′:
a type IIS restriction site in the (+) orientation (S1),
a designed Right sticky end DNA sequence (1*), and
a gene-specific sequence;
a DNA polymerase; and
a chemical buffer suitable for PCR amplification;
thermal cycling the solution between 5 cycles and 60 cycles, with each cycle comprising between 5 seconds and 1 minute at a temperature between 90° C. and 100° C., and between 30 seconds and 2 minutes at a temperature between 55° C. and 72° C.

16. The method of claim 15, wherein a set of gene-specific DNA Blockers are additionally added to the DNA template solution, wherein the region of the DNA template that the Blockers bind overlaps with that of the forward DNA primers by between 4 and 15 nucleotides, and wherein the standard free energy of the forward primer displacing the Blocker at 60° C. in 5 mM Mg²⁺is between 0 kcal/mol and +5 kcal/mol.

17. A method of generating DNA monomers for linear assembly, the method comprising:
obtaining a DNA sample solution that comprises a DNA template;
amplifying the DNA template by multiplex polymerase chain reaction (PCR) amplification, comprising:
adding to the DNA solution:
a set of forward DNA primers comprising, from 5′ to 3′:
a type IIS restriction site in the (+) orientation (S1),
a designed Left sticky end DNA sequence (1), and
a gene-specific sequence;
a set of reverse DNA primers comprising, from 5′ to 3′:
a type IIS restriction site in the (+) orientation (S1),
a designed Right sticky end DNA sequence (1*), and
a gene-specific sequence;
a DNA polymerase; and
a chemical buffer suitable for PCR amplification;
thermal cycling the solution between 5 cycles and 60 cycles, with each cycle comprising between 5 seconds and 1 minute at a temperature between 90° C. and 100° C., and between 30 seconds and 2 minutes at a temperature between 55° C. and 72° C.

18. The method of claim 17, wherein the forward and/or reverse primers further comprise a UMI barcode.

19. The method of claim 17, wherein a set of gene-specific DNA Blockers are additionally added to the DNA template solution, wherein the region of the DNA template that the Blockers bind overlaps with that of the forward DNA primers by between 4 and 15 nucleotides, and wherein the standard free energy of the forward primer displacing the Blocker at 60° C. in 5 mM Mg²⁺is between 0 kcal/mol and +5 kcal/mol.

20. A method for preparing a solution of heterogeneous DNA concatemers, the method comprising:
preparing a set of DNA monomers from a DNA template sample according to the method of any one of claims 17-19;
purifying the monomers to remove unreacted primers and enzymes; and
performing linear DNA assembly according to the method of claim 11 or 12.

21. The method of claim 20, wherein a set of gene-specific DNA Blockers are additionally added to the DNA template solution, wherein the region of the DNA template that the Blockers bind overlaps with that of the forward DNA primers by between 4 and 15 nucleotides, and wherein the standard free energy of the forward primer displacing the Blocker at 60° C. in 5 mM Mg²⁺is between 0 kcal/mol and +5 kcal/mol.

22. The method of claim 20, wherein purifying the monomers comprises using either an affinity column or magnetic beads.

23. A method for targeted nanopore sequencing of gene regions of interest, the method comprising:
obtaining a DNA sample of interest comprising a DNA template;
preparing a set of DNA monomers from the DNA template according to the method of any one of claims 17-19;
purifying the monomers to remove unreacted primers and enzymes;
performing linear DNA assembly according to the method of any one of claims 11-13;
purifying the concatemers to remove unreacted monomers, Type IIS reaction side products, and enzymes;
appending adapters for nanopore sequencing to the purified concatemers;
purifying the adapter-appended concatemers to remove excess adapters and enzymes; and
performing nanopore sequencing.

24. A method for constructing a monomer species comprising, from 5′ to 3′:
a type IIS restriction site in the (+) orientation (S1),
a designed Left sticky end DNA sequence (1),
an insert sequence (A),
a second designed Right sticky end DNA sequence (1*), and
a type IIS restriction site in the (−) orientation (S1*);
wherein at least two different DNA monomers comprise the same Left sticky end DNA sequence, wherein at least two different DNA monomers comprise the same Right sticky end DNA sequence, and wherein the Left sticky end DNA sequence and the Right sticky end DNA sequence are complementary to and can form Watson-Crick base pairs with each other;
the method comprising:
obtaining a solution of double-stranded DNA inserts of interest;
performing a first ligation reaction on a first portion of the solution with a double stranded DNA adaptor comprising:
a type IIS restriction site in the (+) orientation (S1), and
a designed Left sticky end DNA sequence (1);
performing a second reaction ligation reaction on a second portion of the solution with a double stranded DNA adaptor comprising:
a type IIS restriction site in the (+) orientation (S1), and
a designed Right sticky end DNA sequence (1*); and
mixing the products of the first and second ligations reactions in a solution in a chemical buffer conducive to ligation.

25. The method of claim 24, wherein the double-stranded DNA inserts are dA-tailed prior to performing the ligation.

26. A method for targeted nanopore sequencing of gene regions of interest, the method comprising:
obtaining a DNA sample of interest comprising a DNA template;
preparing a set of DNA monomers from the DNA template according to the method of claim 24 or 25;
purifying the monomers to remove unreacted primers and enzymes;
performing linear DNA assembly according to the method of any one of claims 11-13;
purifying the concatemers to remove unreacted monomers, Type IIS reaction side products, and enzymes;
appending adapters for nanopore sequencing to the purified concatemers;
purifying the adapter-appended concatemers to remove excess adapters and enzymes; and
performing nanopore sequencing.

27. The methods of any one of claims 11-26, wherein the step of mixing the DNA monomers further comprises mixing with two single-stranded destructive probes, the first single-stranded destructive probe comprising, from 5′ to 3′,
a type IIS recognition sequence (S1), and
a Left sticky end DNA sequence (1);
and the second single-stranded destructive probe comprising, from 5′ to 3′:
a type IIS recognition sequence (S1), and
the Right sticky end DNA sequence (1*).

28. The method of claim 27, wherein the concentration of the destructive probe is between 1x and 100x of the total concentration of the DNA monomers.

29. The methods of claim 27 or 28, wherein the destructive probes have chemical modifications that prevents restriction digestion.

30. The method of claim 29, wherein the modifications are selected from phosphorothioate-substituted backbone, sugar modified nucleotides (e.g., 2′Fluoro, 2′-OMe), inverted DNA nucleotides, methylated bases, DNA with carbon spacers, or DNA with polyethylene glycol (PEG) spacers.
